(19) United States
(12) Patent Application Publication
(10) Pub. No.: US 2009/0170762 A1
(43) Pub. Date: Jul. 2, 2009

(54) Treatment of Diabetes Related Obesity

(75) Inventors: Peter Raymond Flatt, Antrim (GB); Finbarr Paul Mary O'Harte, Londonderry (GB)

Correspondence Address: K&L Gates LLP, STATE STREET FINANCIAL CENTER, One Lincoln Street, BOSTON, MA, 02111-2950, US

(73) Assignee: UUTech Limited, Coleraine (GB)

(21) Appl. No.: 11/991,639

(22) PCT Filed: Sep. 8, 2006
(86) PCT No.: PCT/EP2006/008780

§ 371 Date: Feb. 18, 2009

Related U.S. Application Data
(60) Provisional application No. 60/715,336, filed on Sep. 8, 2005.

(51) Int. Cl. A61K 38/17; C07K 14/47

(52) U.S. Cl. 514/12; 530/300

(57) ABSTRACT

Peptide analogues and uses are provided for treating and preventing obesity and for treating, preventing and reversing weight gain and related metabolic disease, and promoting weight loss and weight maintenance, by administering a medicament comprising an antagonist of GIP receptor, which is a peptide analogue of GIP.

### FIELD OF THE INVENTION

The present invention relates to the use of peptide analogues of gastric inhibitory peptide (GIP) for the manufacture of a medicament for the treatment of obesity and weight gain, and related metabolic disease. The present invention also relates to certain novel peptide analogues of GIP and pharmaceutical compositions comprising them.

### BACKGROUND

It has been estimated that about one quarter of the US adult population suffers from obesity and over half of the population is overweight. As these numbers continue to climb in the United States and the rest of the world, the health-related costs due to increased incidence of such related diseases as heart disease and diabetes also climb. In 1998, it was reported that the direct economic cost of obesity in the US was $56 billion, a number comparable to the health cost of cigarette smoking (Wolf and Colditz, 1998, Obes. Res. 6:97-106). Methods for treating and preventing obesity and related metabolic disease are therefore highly desirable.

### SUMMARY OF THE INVENTION

A method and use are provided for treating and preventing obesity, preventing weight gain and promoting weight loss in mammals, by administration of a peptide analogue of GIP (gastric inhibitory polypeptide; glucose-dependent insulinotropic polypeptide), which peptide analogue antagonizes the GIP receptor (GIP-R). Certain peptide analogues that antagonize GIP-R are also provided.

The invention provides a method of decreasing or preventing obesity, preventing or ameliorating weight gain and promoting weight loss, increasing insulin sensitivity, improving blood glucose control or decreasing levels of circulating triglycerides, circulating LDL-C or serum cholesterol in a mammal (and corresponding uses) where the method/use includes administering to a mammal a therapeutically effective amount of a medicament comprising a peptide analogue of at least 12 amino acid residues from the N-terminal end of GIP(1-42), wherein the peptide analogue is a GIP antagonist and wherein there is an amino acid substitution or modification at position 3. The amino acid at the 3 position can be substituted by any L-amino acid selected from L-alanine, L-arginine, L-asparagine, L-aspartic acid, L-cysteine, L-glutamine, L-glycine, L-histidine, L-isoleucine, L-leucine, L-lysine, L-methionine, L-phenylalanine, L-proline, L-serine, L-threonine, L-tryptophan, L-tyrosine and L-valine. The amino acid at the 3 position (or, indeed, the optional 1 and 2 positions mentioned below) can be substituted by any other L- or D-amino acid other than those commonly encountered in the genetic code, including beta amino acids such as beta-alanine and omega amino acids such as 3-amino propionic, 4-amino butyric, etc, ornithine, citrulline, homoarginine, t-butyl alanine, t-butyl glycine, N-methyl isoleucine, phenylglycine, cyclohexylalanine, norleucine, cysteic acid, and methionine sulfoxide. The amino acid at the 3 position can be substituted by lysine, serine, proline, hydroxyproline, alanine, phenylalanine, tryptophan, tyrosine, 4-amino butyric acid (Abu), amino isobutyric acid (Aib), or sarcosine. For instance, the peptide analogues can include, but are not limited to, (Lys³)GIP, (Ser³)GIP, (Pro³)GIP, (Hyp³)GIP, (Ala³)GIP, (Phe³)GIP, (Trp³)GIP, (Tyr³)GIP, (Abu³)GIP or (Sar³)GIP. The amino acid substitution at position 3 can include a D-amino acid substitution at position 3. The amino acid at the 3 position can be substituted by any D-amino acid selected from by D-arginine, D-asparagine, D-aspartic acid, D-cysteine, D-glutamine, D-glycine, D-histidine, D-isoleucine, D-leucine, D-lysine, D-methionine, D-phenylalanine, D-proline, D-serine, D-threonine, D-tryptophan, D-tyrosine and D-valine. The, or each, D-amino acid substitution can comprise replacement of the L-amino acid with its corresponding D-amino acid. Alternatively, the, or each, D-amino acid substitution can comprise replacement of the L-amino acid with any other D-amino acid. The amino acid at the 3 position can be modified by the substitution of a short chain C2-5 radical for one of the hydrogens on the nitrogen of Glu or by a short chain C2-5 radical for both of the hydrogens on the nitrogen of Glu.

The peptide analogues used in the methods and uses can further include an amino acid substitution or an amino acid modification at one or both of positions 1 or 2 and can further include an amino acid substitution or modification at positions 1 or 2, for instance, a D-amino acid substitution at position 1 or a D-amino acid substitution at position 2. The, or each, D-amino acid substitution can comprise replacement of the L-amino acid with its corresponding D-amino acid. Alternatively, the, or each, D-amino acid substitution at one or both of positions 1 and 2 can comprise replacement of the L-amino acid with any other D-amino acid, for example selected from by D-alanine, D-arginine, D-asparagine, D-aspartic acid, D-cysteine, D-glutamic acid, D-glutamine, D-glycine, D-histidine, D-isoleucine, D-leucine, D-lysine, D-methionine, D-phenylalanine, D-proline, D-serine, D-threonine, D-tryptophan, D-tyrosine and D-valine. The, or each, L-amino acid substitution at one or both of positions 1 and 2 can comprise replacement of the L-amino acid with any other L-amino acid, for example selected from by L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine and L-valine. The amino acid in the 2 position can also be substituted by lysine, serine, proline, hydroxyproline, alanine, phenylalanine, tryptophan, tyrosine, 4-amino butyric acid (Abu), amino isobutyric acid (Aib), or sarcosine.

The peptide analogues used in the methods can be further modified at position 1 by N-terminal alkylation, N-terminal acetylation, N-terminal C₆₋₂₀ acylation, the addition of an N-terminal isopropyl group, the addition of an N-terminal pyroglutamic acid, or the addition of an N-terminal polyethylene glycol (PEG) molecule. For instance, the peptide analogues can include, but are not limited to, N-Ac(Lys³)GIP, N-Ac(Ser³)GIP, N-Ac(Pro³)GIP, N-Ac(Hyp³)GIP, N-Ac(Ala³)GIP, N-Ac(Phe³)GIP, N-Ac(Trp³)GIP, N-Ac(Tyr³)GIP, N-Ac(Abu³)GIP or N-Ac(Sar³)GIP.

The peptide analogues used in the methods can include a modification by acyl radical addition, optionally a fatty acid addition, at an epsilon amino group of at least one lysine residue, for instance, the modification can be the linking of a C-8 octanoyl group, C-10 decanoyl group, C-12 lauroyl group, C-14 myristoyl group, C-16 palmitoyl group, C-18 stearoyl group, or C-20 acyl group to the epsilon amino group of a lysine residue, for instance, the linking of a C-16 palmitoyl group to a lysine residue chosen from the group consisting of Lys¹⁶, Lys³⁰, Lys³², Lys³³ and Lys³⁷. For instance, the peptide analogues can include, but are not limited to,

(Lys³)GIP(LysPAL¹⁶), (Lys³)GIP(LysPAL³⁷), N-Ac(Lys³)GIP(LysPAL¹⁶), N-
Ac(Lys³)GIP(LysPAL³⁷), (Ser³)GIP(LysPAL¹⁶), (Ser³)GIP(LysPAL³⁷), N-
Ac(Ser³)GIP(LysPAL¹⁶), N-Ac(Ser³)GIP(LysPAL³⁷), (Pro³)GIP(LysPAL¹⁶),
(Pro³)GIP(LysPAL³⁷), N-Ac(Pro³)GIP(LysPAL¹⁶), N-Ac(Pro³)GIP(LysPAL³⁷),
(Hyp³)GIP(LysPAL¹⁶), (Hyp³)GIP(LysPAL³⁷), N-AC(Hyp³)GIP(LysPAL¹⁶), N-
Ac(Hyp³)GIP(LysPAL³⁷), (Ala³)GIP(LysPAL¹⁶), (Ala³)GIP(LysPAL³⁷), N-
Ac(Ala³)GIP(LysPAL¹⁶), N-Ac(Ala³)GIP(LysPAL³⁷), (Phe³)GIP(LysPAL¹⁶),
(Phe³)GIP(LysPAL³⁷), N-Ac(Phe³)GIP(LysPAL¹⁶), N-Ac(Phe³)GIP(LysPAL³⁷),
(Trp³)GIP(LysPAL¹⁶), (Trp³)GIP(LysPAL³⁷), N-Ac(Trp³)GIP(LysPAL¹⁶), N-
Ac(Trp³)GIP(LysPAL³⁷), (Tyr³)GIP(LysPAL¹⁶), (Tyr³)GIP(LysPAL³⁷), N-
Ac(Tyr³)GIP(LysPAL¹⁶), N-Ac(Tyr³)GIP(LysPAL³⁷), (Abu³)GIP(LysPAL¹⁶),
(Abu³)GIP(LysPAL³⁷), N-Ac(Abu³)GIP(LysPAL¹⁶), N-Ac(Abu³)GIP(LysPAL³⁷),
(Aib³)GIP(LysPAL¹⁶), (Aib³)GIP(LysPAL³⁷), N-Ac(Aib³)GIP(LysPAL¹⁶), N-
Ac(Aib³)GIP(LysPAL³⁷), (Sar³)GIP(LysPAL¹⁶), (Sar³)GIP(LysPAL³⁷), N-
Ac(Sar³)GIP(LysPAL¹⁶), or N-Ac(Sar³)GIP(LysPAL³⁷).

Any of the peptide analogues can be covalently attached to a polyethylene glycol (PEG) molecule.

The medicament can also include a pharmaceutically acceptable carrier. The peptide analogues used in the medicaments can be in the form of a pharmaceutically acceptable salt, such as a pharmaceutically acceptable acid addition salt. The medicaments can also include an agent having an antidiabetic effect.

The peptide analogues described herein can be used for decreasing or preventing obesity, preventing weight gain and promoting weight loss, increasing insulin sensitivity, improving blood glucose control, decreasing levels of circulating triglycerides, decreasing levels of circulating LDL-C, or decreasing levels of serum cholesterol.

The peptide analogues described herein can be used as a medicament for decreasing or preventing obesity, preventing weight gain and promoting weight loss, increasing insulin sensitivity, improving blood glucose control, decreasing levels of circulating triglycerides, decreasing levels of circulating LDL-C, or decreasing levels of serum cholesterol.

The peptide analogues can also include the addition of linkers or residues to the N-terminal or C-terminal ends of the protein.

The peptide analogues can be used to screen compounds for their potential use as agonists or antagonists of the GIP receptor.

The peptide analogues can also be used to cause stem cells to differentiate into beta cells, to provide cell or replacement therapy for diabetes.

The invention includes use of a peptide analogue of GIP in the manufacture of a medicament for the treatment of one or more of: decreasing or preventing obesity, preventing weight gain and promoting weight loss, improving blood glucose control, increasing insulin sensitivity, or decreasing levels of circulating triglycerides, circulating LDL-C or serum cholesterol. The peptide analogue has at least 12 amino acid residues from the N-terminal end of GIP(1-42) (optionally human GIP(1-42)) and wherein there is an amino acid substitution or modification at position 3. The peptide analogue can also include an amino acid substitution and/or amino acid modification at one or both of positions 1 and 2, such as a D-amino acid substitution at position 1 or a D-amino acid substitution at position 2. The amino acid in the 2 or 3 position can be substituted by lysine, serine, proline, hydroxyproline, alanine, phenylalanine, tryptophan, tyrosine, 4-amino butyric acid (Abu), amino isobutyric acid (Aib), or sarcosine. The peptide analogue can be covalently attached to a polyethylene glycol (PEG) molecule. The peptide analogue can also be in the form of a pharmaceutically acceptable salt, for instance, a pharmaceutically acceptable acid addition salt.

The peptide analogues, such as (Lys³)GIP(LysPAL¹⁶); (Lys³)GIP(LysPAL³⁷), N-Ac(Lys³)GIP(LysPAL¹⁶), N-Ac(Lys³)GIP(LysPAL³⁷), (Ser³)GIP(LysPAL¹⁶), (Ser³)GIP(LysPAL³⁷), N-Ac(Ser³)GIP(LysPAL¹⁶), N-Ac(Ser³)GIP(LysPAL³⁷), (Pro³)GIP(LysPAL¹⁶), (Pro³)GIP(LysPAL³⁷), N-Ac(Pro³)GIP(LysPAL¹⁶), N-Ac(Pro³)GIP(LysPAL³⁷), (Hyp³)GIP(LysPAL¹⁶), (Hyp³)GIP(LysPAL³⁷), N-Ac(Hyp³)GIP(LysPAL¹⁶), N-Ac(Hyp³)GIP(LysPAL³⁷), (Ala³)GIP(LysPAL¹⁶), (Ala³)GIP(LysPAL³⁷), N-Ac(Ala³)GIP(LysPAL¹⁶), N-Ac(Ala³)GIP(LysPAL³⁷), (Phe³)GIP(LysPAL¹⁶), (Phe³)GIP(LysPAL³⁷), N-Ac(Phe³)GIP(LysPAL¹⁶), N-Ac(Phe³)GIP(LysPAL³⁷), (Trp³)GIP(LysPAL¹⁶), (Trp³)GIP(LysPAL³⁷), N-Ac(Trp³)GIP(LysPAL¹⁶), N-Ac(Trp³)GIP(LysPAL³⁷), (Tyr³)GIP(LysPAL¹⁶), (Tyr³)GIP(LysPAL³⁷), N-Ac(Tyr³)GIP(LysPAL¹⁶), N-Ac(Tyr³)GIP(LysPAL³⁷), (Abu³)GIP(LysPAL¹⁶), (Abu³)GIP(LysPAL³⁷), N-Ac(Abu³)GIP(LysPAL¹⁶), N-Ac(Abu³)GIP(LysPAL³⁷), (Aib³)GIP(LysPAL¹⁶), (Aib³)GIP(LysPAL³⁷), N-Ac(Aib³)GIP(LysPAL¹⁶), N-Ac(Aib³)GIP(LysPAL³⁷), (Sar³)GIP(LysPAL¹⁶), (Sar³)GIP(LysPAL³⁷), N-Ac(Sar³)GIP(LysPAL¹⁶), or N-Ac(Sar³)GIP(LysPAL³⁷) can be used in the manufacture of a medicament for decreasing or preventing obesity, preventing weight gain and promoting weight loss, increasing insulin sensitivity, decreasing levels of circulating triglycerides, decreasing levels of circulating LDL-C, or decreasing levels of serum cholesterol. In such uses, the peptide analogue can be covalently attached to a polyethylene glycol (PEG) molecule.

Peptide analogues for use in the invention comprise peptide analogues of GIP(1-42), comprising at least 12 amino acids from the N-terminal end of GIP(1-42) (SEQ ID NO:1). They comprise peptide analogues of GIP(1-42) (SEQ ID NO:1), comprising at least 12 amino acid residues from the N-terminal end of GIP(1-42) and having an amino acid substitution or modification at position 3 (Glu³), (such as, for instance, substitution of Glu³ with serine, proline, hydroxyproline, lysine, tyrosine, alanire, phenylalanine, serine, alanine, 4-amino butyric acid (Abu), amino isobutyric acid (Aib), sarcosine or tryptophan). The peptide analogues also include analogues comprising at least 12 amino acid residues from the N-terminal end of GIP(1-42), and having an amino acid substitution or modification at Glu³ (such as, for instance, substitution of Glu³ with proline, hydroxyproline, lysine, tyrosine, phenylalanine, serine, alanine, 4-amino butyric acid (Abu), amino isobutyric acid (Aib), sarcosine or tryptophan) and further having an amino acid modification at one or more of amino acid residues 1 and 2 (such as N-terminal alkylation, N-terminal acetylation, N-terminal acylation, the addition of an N-terminal isopropyl group, the addition of an N-terminal pyroglutamic acid or the addition of an N-terminal polyethylene glycol (PEG) molecule).

The peptide analogues can also be modified by conversion of one or more bonds between the first, second and third residues to a psi [CH₂NH] bond, or to a stable isotere bond.

The peptide analogues used in the methods can include a modification by an acyl radical, optionally a fatty acid, addition at an epsilon amino group of at least one lysine residue, for instance, the modification can be the linking of a C-8 octanoyl group, C-10 decanoyl group, C-12 lauroyl group, C-14 myristoyl group, C-16 palmitoyl group, C-18 stearoyl group, or C-20 acyl group to the epsilon amino group of a lysine residue, for instance, the linking of a C-16 palmitoyl group to a lysine residue chosen from the group consisting of Lys¹⁶, Lys³⁰, Lys³², Lys³³ and Lys³⁷.

For instance, the peptide analogue can be a peptide analogue of GIP(1-42) (SEQ ID NO:1), wherein the analogue comprises: a base peptide consisting of one of the following: GIP(1-12), GIP(1-13), GIP(1-14), GIP(1-15), GIP(1-16), GIP(1-17), GIP(1-18), GIP(1-19), GIP(1-20), GIP(1-21), GIP(1-22), GIP(1-23), GIP(1-24), GIP(1-25), GIP(1-26), GIP(1-27), GIP(1-28), GIP(1-29), GIP(1-30), GIP(1-31), GIP(1-32), GIP(1-33), GIP(1-34), GIP(1-35), GIP(1-36), GIP(1-37), GIP(1-38), GIP(1-39), GIP(1-40), GIP(1-41) and GIP(1-42); which possesses an amino acid modification or an amino acid substitution at Glu³, and which may possess one or more of the following further amino acid substitutions or amino acid modifications: (a) an amino acid substitution at one or more of the residues, for example, one or more of positions 1 and 2, (b) an amino acid substitution of lysine for one or more or the residues, (c) a modification by acyl radical, optionally fatty acid, addition at an epsilon amino group of at least one lysine residue, (d) a modification by N-terminal alkylation, N-terminal acetylation, N-terminal acylation, the addition of an N-terminal isopropyl group, the addition of an N-terminal pyroglutamic acid, and the addition of an N-terminal polyethylene glycol (PEG) molecule, for example, N-terminal acetylation or (e) an amino acid modification at one or more of the residues, for example, one or more of positions 1 and 2. The peptide analogue can be modified by fatty acid addition at an epsilon amino group of at least one lysine residue, such as by the linking of a C-16 palmitate group to the epsilon amino group of a lysine residue, such as lysine residue Lys¹⁶ or lysine residue Lys³⁷.

Any of the peptide analogues described herein can also be non-human derived versions of the GIP protein. For instance, the peptide analogues can be analogues of the GIP protein as it is found in rat, mouse, hamster, sheep, cow, pig, goat, dog, cat, etc.

The invention includes a peptide analogue selected from (Lys³)GIP(LysPAL¹⁶), (Lys³)GIP(LysPAL³⁷), N-Ac(Lys³)GIP(LysPAL¹⁶), N-Ac(Lys³)G(LysPAL³⁷), (Ser³)GIP(LysPAL¹⁶), (Ser³)GIP(LysPAL³⁷), N-Ac(Ser³)GIP(LysPAL¹⁶), N-Ac(Ser³)GIP(LysPAL³⁷), (Pro³)GIP(LysPAL¹⁶), (Pro³)GIP(LysPAL³⁷), N-Ac(Pro³)GIP(LysPAL¹⁶), N-Ac(Pro³)GIP(LysPAL³⁷), (Hyp³)GIP(LysPAL¹⁶), (Hyp³)GIP(LysPAL³⁷), N-Ac(Hyp³)GIP(LysPAL¹⁶), N-Ac(Hyp³)GIP(LysPAL³⁷), (Ala³)GIP, (Ala³)GIP(LysPAL¹⁶), (Ala³)GIP(LysPAL³⁷), N-Ac(Ala³)GIP(LysPAL¹⁶), N-Ac(Ala³)GIP(LysPAL³⁷), (Phe³)GIP(LysPAL¹⁶), (Phe³)GIP(LysPAL³⁷), N-Ac(Phe³)GIP(LysPAL¹⁶), N-Ac(Phe³)GIP(LysPAL³⁷), (Trp³)GIP(LysPAL¹⁶), (Trp³)GIP(LysPAL³⁷), N-Ac(Trp³)GIP(LysPAL¹⁶), N-Ac(Trp³)GIP(LysPAL³⁷), (Tyr³)GIP(LysPAL¹⁶), (Tyr³)GIP(LysPAL³⁷), N-Ac(Tyr³)GIP(LysPAL¹⁶), N-Ac(Tyr³)GIP(LysPAL³⁷), (Abu³)GIP(LysPAL¹⁶), (Abu³)GIP(LysPAL³⁷), N-Ac(Abu³)GIP(LysPAL¹⁶), N-Ac(Abu³)GIP(LysPAL³⁷), (Aib³)GIP(LysPAL¹⁶), (Aib³)GIP(LysPAL³⁷), N-Ac(Aib³)GIP(LysPAL¹⁶), N-Ac(Aib³)GIP(LysPAL³⁷), (Sar³)GIP(LysPAL¹⁶), (Sar³)GIP(LysPAL³⁷), N-Ac(Sar³)GIP(LysPAL¹⁶) and N-Ac(Sar³)GIP(LysPAL³⁷). Optionally, the peptide analogue can be selected from (Ala³)GIP, (Ala³)GIP(LysPAL¹⁶), (Ala³)GIP(LysPAL³⁷), N-Ac(Ala³)GIP(LysPAL¹⁶), N-Ac(Ala³)GIP(LysPAL³⁷), (Pro³)GIP(LysPAL¹⁶), (Pro³)GIP(LysPAL³⁷), N-Ac(Pro³)GIP(LysPAL¹⁶), N-Ac(Pro³)GIP(LysPAL³⁷), (Hyp³)GIP(LysPAL¹⁶), (Hyp³)GIP(LysPAL³⁷), N-Ac(Hyp³)GIP(LysPAL¹⁶) and N-Ac(Hyp³)GIP(LysPAL³⁷). The peptide analogue can selected from the group comprising (Ala³)GIP, (Pro³)GIP(LysPAL¹⁶) and (Hyp³)GIP(LysPAL¹⁶). Any of the peptide analogues described herein can be included in a pharmaceutical composition. Such a pharmaceutical composition includes a pharmaceutically acceptable carrier. The peptide analogues can be in the form of a pharmaceutically acceptable salt, and/or a pharmaceutically acceptable acid addition salt.

The peptide analogues can be combined with other treatment regimens, for instance, the peptide analogues can be combined with other antidiabetic agents, such as biguanides (such as, but not limited to, metformin), sulphonylureas (such as, but not limited to, acetohexamide, chlorpropamide, tolbutamide, tolazamide, glimepiride, gliclazide, glipizide, glyburide, glibenclamide), thiazolidinediones (also called glitazones) (such as, but not limited to, pioglitazone (e.g., pioglitazone hydrochloride), rosiglitazone (rosiglitazone maleate), troglitazone), meglitinides (such as, but not limited to, nateglinide, repaglinide), alpha-glucosidase inhibitors (such as, but not limited to, acarbose, miglitol), incretin mimetics (such as, but not limited to, exenatide), amylinomimetics (such as, but not limited to, pramlintide acetate). The peptide analogues can also be combined with other peptide molecules, such as GLP-1, or analogues of such peptide molecules.

The peptide analogues disclosed herein can also be combined with other anti-obesity, lipid lowering and metabolic syndrome treatments, such as, but not limited to, cannabinoid antagonists, lipase inhibitors, dual serotonin and norepinephrin reuptake inhibitors, beta-3 agrenergic agonists, cholecystokinin agonists, ciliary neurotrophic factor (CNTF) agonists, leptin antagonists, lipid metabolism modulators, or other treatments such as diets and dietary formulations.

### BRIEF DESCRIPTION OF THE DRAWINGS

FIGS. 1A and 1B are a pair of line graphs showing the effects of daily (Pro³)GIP administration (▴) on food intake (FIG. 1A, y-axis) and body weight (FIG. 1B, y-axis) of ob/ob mice over time (x-axis), relative to saline-treated controls (□). Parameters were measured for 5 days prior to, 60 days during (indicated by black bar) treatment with saline or (Pro³)GIP (25 nmol/kg bw/day). Values are mean±SEM for 7-8 mice.

FIGS. 2A-2C are a pair of line graphs and a bar chart, respectively, showing the effects of daily (Pro³)GIP administration (▴, black bar) on non-fasting plasma glucose (FIG. 2A), plasma insulin (FIG. 2B) and glycated haemoglobin concentrations (FIG. 2C) of ob/ob mice, relative to saline-treated controls (□, white bar). Plasma glucose and insulin concentrations were measured for 5 days prior to, 60 days during treatment (indicated by black bar) with saline or (Pro³)GIP (25 nmol/kg bw/day). Glycated haemoglobin concentrations were assessed on day 60. Values are mean±SEM for 7-8 mice. *P<0.05, **P<0.01, ***P<0.001 compared with saline group.

FIGS. 3A-3D are two line graphs (FIGS. 3A and 3C) and two bar graphs (FIGS. 3B and 3D), showing the effects of daily (Pro³)GIP administration (A, black bars) on glucose tolerance and plasma insulin response to glucose in ob/ob mice, relative to controls (o, white bars). Tests were conducted after daily treatment with (Pro³)GIP (25 nmol/kg body weight/day) for 60 days. Glucose (18 mmol/kg body weight) was administered at the time indicated by the arrow. Plasma glucose and plasma insulin values are shown in FIGS. 3A and 3C. Plasma glucose AUC and plasma insulin AUC values for 0-60 min post injection are also shown (FIGS. 3B and 3D). Values are mean±SEM for 8 mice. *P<0.05, **P<0.01 and ***P<0.001 compared with saline group.

FIGS. 4A-4D are two line graphs (FIGS. 4A and 4C) and two bar graphs (FIGS. 4B and 4D), showing the effects of daily (Pro³)GIP administration (A, black bars) on metabolic response to native GIP in ob/ob mice, relative to controls (o, white bars). Tests were conducted after daily treatment with (Pro³)GIP (25 nmoles/kg body weight/day) for 60 days. Glucose (18 mmol/kg body weight) in combination with native GIP (25 nmoles/kg body weight) was administered at the time indicated by the arrow. Plasma glucose and plasma insulin values are shown in FIGS. 4A and 4C. Plasma glucose AUC and plasma insulin AUC values for 0-60 min post injection are also shown (FIGS. 4B and 4D). Values are mean±SEM for 7-8 mice. *P<0.05 and **P<0.01 compared with saline group.

FIGS. 5A-5D are two line graphs (FIGS. 5A and 5C) and two bar graphs (FIGS. 5B and 5D), showing the effects of daily (Pro³)GIP administration (▴, black bars) on glucose and insulin responses to feeding in 18 hour fasted ob/ob mice, relative to controls (□, white bars). Tests were conducted after daily treatment with (Pro³)GIP (25 nmol/kg body weight/day) or saline for 60 days. The arrow indicates the time of feeding (15 minutes). Plasma glucose and plasma insulin values are shown in FIGS. 5A and 5C. Plasma glucose AUC and plasma insulin AUC values for 0-105 minutes post-feeding are also shown (FIGS. 5B and 5D). Values are mean±SEM for 7-8 mice. *P<0.05, **P<0.01 and ***P<0.001 compared with saline group.

FIGS. 6A-6D are two line graphs (FIGS. 6A and 6C) and two bar graphs (FIGS. 6B and 6D), showing the effects of daily (Pro³)GIP administration (▴, black bars) on insulin sensitivity in ob/ob mice, relative to controls (□, white bars). Tests were conducted after daily treatment with (Pro³)GIP (25 mmol/kg body weight/day) or saline for 60 days. Insulin (50 U/kg body weight) was administered by intraperitoneal injection at the time indicated by the arrow. Plasma glucose and plasma insulin values are shown in FIGS. 6A and 6C. Plasma glucose AUC and plasma insulin AUC values for 0-60 min post-injection are also shown (FIGS. 6B and 6D). Values are mean±SEM for 7-8 mice. FIG. 6A displays data as % of basal values and FIG. 6B as whole numbers. *P<0.05 and **P<0.01 compared with saline group.

FIGS. 7A and 7B are a pair of bar charts showing the effects of daily (Pro³)GIP administration on pancreatic weight (FIG. 7A) and insulin content (FIG. 7B). Parameters were measured after daily treatment with (Pro³)GIP (25 nmol/kg body weight/day, black bars) or saline (white bars) for 60 days. Values are mean±SEM for 7-8 mice. **P<0.01 compared with saline group.

FIGS. 8A-8D are a set of four bar graphs showing the effects of daily (Pro³)GIP administration on lipid profile in (ob/ob) mice on circulating triglyceride concentrations (FIG. 8A), cholesterol levels (FIG. 8B), LDL-C levels (FIG. 8C) and HDL-C levels (FIG. 8D). Parameters were measured after daily treatment with (Pro³)GIP (25 nmol/kg body weight/day; black bars) or saline (white bars) for 11 days. Cross-hatched bars correspond to levels of these compounds in age-matched normal lean control mice. LDL-C was calculated using the Friedewald Equation. Values are mean±SEM for 7-8 mice. *P<0.05 compared with saline group.

FIGS. 9A-9D are a set of four line graphs showing the effects of daily (Pro³)GIP administration on body weight of normal (TO) mice fed high fat (FIG. 9A), cafeteria (FIG. 9B), high carbohydrate (FIG. 9C), and normal (FIG. 9D) diets. Body weight was measured for 5 days prior to and 90 days during treatment with saline (Δ) or (Pro³)GIP (25 nmol/kg bw/day; ▪) in groups of normal mice given access ad libitum to high fat diet, high carbohydrate diet, cafeteria diet and normal rodent diet. Values are means±SEM for 8-10 mice. *P<0.05 and **P<0.01 compared with saline (non-treated) group.

FIGS. 10A and 10B are a pair of bar graphs showing the effects of GIP analogs ((Ala³)GIP, (Lys³)GIP, (Phe³)GIP, (Trp³)GIP, (Tyr³)GIP and (Pro³)GIP) on antihyperglycaemic and insulin releasing actions relative to native GIP, when administered with glucose to ob/ob mice. Plasma glucose AUC (FIG. 10A) and plasma insulin AUC (FIG. 10B) values for 0-60 minutes post-injection are shown. Data are expressed as mean±S.E. for 8 mice. *p<0.05, **p<0.01, ***p<0.001 compared with glucose alone. ^{Δ}p<0.05, ^{ΔΔ}p<0.01, ^{ΔΔΔ}p<0.001 compared with native GIP.

FIGS. 11A and 11B are a pair of line graphs showing glucose tolerance in ob/ob mice following 14 once-daily injections of saline (□), (Pro³)GIP(▴) or (Hyp³)GIP () (FIG. 11A) or saline (□), (Pro³)LysPAL¹⁶GIP (▴) and (Hyp³)LysPAL¹⁶GIP () (FIG. 11B). Mice were administered glucose (18 mmol/kg body wt) or peptide analogue (25 nmoles/kg body weight) once daily for 14 days, and glucose was measured after injection. The time of injection is indicated by the arrows. Values are mean±S.E.M. for eight mice. *P<0.05, **P<0.01, ***P<0.001 compared to saline control.

FIGS. 12A and 12B are a pair of line graphs showing plasma insulin response in ob/ob mice following 14 once-daily injections of saline (□), (Pro³)GIP (▴) or (Hyp³)GIP () (FIG. 12A) or saline (□), (Pro³)LysPAL¹⁶GIP (▴) and (Hyp³)LysPAL¹⁶GIP () (FIG. 12B). Mice were administered glucose (18 mmol/kg body wt) or peptide analogue (25 nmoles/kg body weight) once daily for 14 days, and glucose was measured after injection. The time of injection is indicated by the arrows. Values are mean±S.E.M. for eight mice. *P<0.05, **P<0.01 compared to saline control.

FIGS. 13A and 13B are a pair of line graphs showing insulin sensitivity in ob/ob mice following 14 once-daily injections of saline (□), (Pro³)GIP (▴) or (Hyp³)GIP () (FIG. 13A) or saline (□), (Pro³)LysPAL¹⁶GIP (▴) and (Hyp³)LysPAL¹⁶GIP () (FIG. 13B). Mice were administered intraperitoneal insulin (50 U/kg body wt) or peptide analogue (25 nmoles/kg body weight) once daily for 14 days, and glucose was measured after injection. The time of injection is indicated by the arrows. Values are mean±S.E.M. for eight mice. **P<0.01, ***P<0.001 compared to saline control.

FIGS. 14A and B are a pair of line graphs showing the effects of weekly energy consumption (A & B) of Swiss TO mice fed high fat and ‘cafeteria’ diets respectively. Parameters were measured at 3-4 daily intervals during the 120-day treatment period. Mice were recruited into the study at 6-8 weeks of age. Control animals received standard rodent maintenance diet, 12.99 kj/g, (Harlan, UK) ad libitum. High fat diet groups received a special diet composed of 45% fat, 20% protein and 35% carbohydrate (26.15 kj/g) available ad libitum. (Special Diet Services, Essex, UK). Cafeteria-fed animals received standard rodent maintenance diet, 12.99 kj/g, (Harlan, UK) ad libitum, alongside a six-day rotation of the following food pairs; tuna fish and Pringles, peanut butter and chocolate digestives, Madeira cake and milk chocolate, cereal and luncheon meat, sausages and corned beef and cheese and marzipan. Both high fat and ‘cafeteria’ diet animals received once daily intraperitoneal injections of saline or (Pro³)GIP (25 nmol/kg body weight). Control animals received daily saline injections. Values are mean±S.E.M for 9 mice. *P<0.05, **P<0.01 and ***P<0.001 compared with control group. ΔP<0.05, ΔΔP<0.01 and ΔΔΔP<0.001 compared with (Pro³)GIP treated group.

FIGS. 15A and B are a pair of line graphs showing the effects of daily (Pro³)GIP administration on body weight (A) and food intake (B) of Swiss TO mice fed a high fat diet for 160 days prior to commencement of treatment. Parameters were measured at 3-4 daily intervals during a 60 day treatment period, which was preceded by a 160 days of feeding high fat diet. Mice were initially recruited into the study at 6-8 weeks of age. Control animals received standard rodent maintenance diet, 12.99 kj/g, (Harlan, UK) ad libitum. The animals were maintained on the respective diet for the duration of the study. High fat diet comprised 45% fat, 20% protein and 35% carbohydrate (26.15 kj/g) and was available ad libitum. (Special Diet Services, Essex, UK). High fat diet animals received once daily intraperitoneal injection of saline or with (Pro³)GIP (25 nmol/kg body weight) for 60 days. Values are mean±S.E.M for 12 mice. *P<0.05, **P<0.0 and ***P<0.001 compared with control group. ΔP<0.05, ΔΔP<0.01 and ΔΔΔP<0.001 compared with (Pro³)GIP treated group.

FIGS. 16A and B are a pair of line graphs showing the effects of daily (Pro³)GIP administration on (A) food intake, (B) body weight of 5-7 weeks-old ob/ob mice. Parameters were measured for 5 days prior to, 60 days during (indicated by black bar) treatment with saline or (Pro³)GIP (25 nmol/kg body weight/day). Animals were fed a standard rodent maintenance diet, 12.99 kj/g, (Harlan, UK) ad libitum. Values are mean±SEM for groups of 7-8 mice.

### DETAILED DESCRIPTION

Peptide analogues of GIP, which antagonize the GIP receptor, can be used to treat and prevent obesity and weight gain, promote weight loss and improve obesity-related metabolic disease in mammals. Peptide analogues of GIP capable of antagonizing the GIP-R are provided, along with methods of treatment. The peptide analogues can also be used in methods to improve lipid profile, lower plasma triglycerides and cholesterol, and reduce the risk of cardiovascular disease, especially in individuals with obesity associated with metabolic syndrome and diabetes.

Methods are provided herein for treating and preventing obesity and weight gain, and for promoting weight loss and weight maintenance. Methods are provided for using peptide analogues of GIP to decrease non-fasting plasma glucose and insulin levels, non-fasting plasma insulin levels, intraperitoneal glucose load, pancreatic insulin content, levels of circulating triglycerides and LDL-C, and serum cholesterol levels. The peptide analogues can also be used to increase insulin sensitivity and treat metabolic syndrome.

Glucose-dependent insulinotropic polypeptide (gastric inhibitory polypeptide; GIP) is a potent insulinotropic hormone of the enteroinsular axis and augments glucose stimulated insulin secretion. GIP also exerts effects at extrapancreatic sites and plays a role in lipid physiology, with elevated levels being associated with obesity. The peptide analogues act as antagonists of the GIP receptor.

As used herein, an “antagonist” is a peptide analogue of GIP, which inhibits, inactivates, blocks or decreases the biological activity triggered by the GIP receptor, or otherwise inhibits, inactivates, blocks or decreases the biological activity shown by native GIP.

GIP has been suggested as having a role in obesity. It has been shown that obese diabetic (ob/ob) mice are noted for intestinal K-cell hyperplasia and markedly elevated concentrations of intestinal and circulating GIP (Flatt, P. R. et al., 1983, Diabetes 32:433-435; Flatt, P. R. et al., 1984, J. Endocrinol. 101:249-256; Bailey, C. J. et al., 1986, Acta Endocrinol. (Copenh) 112:224-229). More notably ob/ob mice cross-bred to genetically knockout GIP-R function displayed decreased body weight gain and significant amelioration of both adiposity and insulin resistance (Miyawaki, K et al., 2002, Nat. Med. 8:738-742). However, these findings may not be entirely predictive because an inherent problem with genetic knockout is undoubtedly the life-long opportunity for compensatory metabolic adaptations.

The most widely accepted role of GIP is potentiation of glucose-induced insulin secretion from pancreatic beta-cells. GIP acts together with GLP-1 to account for the major part of the total incretin effect observed after nutrient ingestion (Green, B. D. et al., 2004, Curr. Pharm. Des. 10:3651-3662). GIP exerts these effects through binding to specific beta-cell receptors, GIP-receptor (GIP-R), causing adenylyl cyclase release. However, the GIP-R is expressed in many tissues including the pancreatic islets, adipose tissue and brain. The potent stimulation of GIP secretion after high fat feeding suggests involvement of GIP in fat metabolism (Kwasowski, P. et al., 1985, Biosci. Rep. 5:701-705). Furthermore, plasma GIP concentrations have been reported to be elevated in obesity-induced type 2 diabetes and obese diabetic (ob/ob) mice (Flatt, P. R. et al., 1983, Diabetes 32:433-435). Functional GIP receptors are present on adipocytes and experimental evidence in mice demonstrates elimination of GIP signaling can prevent obesity (Miyawaki, K et al., 2002, Nat. Med. 8:738-742).

Studies have demonstrated that incretin analogues have strong antidiabetic potential (Green, B. D. et al., 2004, Curr. Pharm. Des. 10:3651-3662), and have revealed that continuous intravenous infusion of GLP-1 has antidiabetic action. However, such a method seems too invasive and cumbersome to have broad clinical application. Moreover, continuous peptide infusion has shown a less pronounced insulin response for GIP compared to a bolus administration (Meier, J. J. et al., 2003, Metabolism 52:1579-1585). Furthermore, a number of pharmnacokinetic and pharmacodynamic limitations thwart the use of incretin hormones as antidiabetic agents (Green, B. D. et al., 2004, Curr. Pharm. Des. 10:3651-3662). These include; their peptidic nature, rapid inactivation by the ubiquitous enzyme dipetidyl peptidase IV (DPP IV) and renal filtration. Some attempts have been made to circumvent their deleterious action profile including N-terminal modification or introduction of fatty acid chains or chemical linkers.

As shown herein, daily injections over 60-90 days of the stable and specific GIP-R antagonist (Pro³)GIP (Gault, V. A., O'Harte, F. P. N, 2002, Biochem. Biophys. Res. Commun. 290:1420-1426) can be used to chemically ablate the GIP-R and evaluate the role of endogenous circulating GIP in obesity-diabetes as manifested in ob/ob mice and animal models of diet-induced obesity and insulin resistance. In addition, a decrease in triglyceride levels was seen. The results provide clear evidence that sustained GIP-R antagonism can provide a novel means of treating obesity-driven forms of glucose intolerance and type 2 diabetes with correction of the many associated metabolic abnormalities in both genetic and dietary induced animal models.

The data presented herein show that chemical ablation of GIP receptor in genetic and dietary induced animal models of obesity-diabetes can be accomplished over the long term using a stable and specific GIP-R antagonist, (Pro³)GIP. Young (5-7 weeks) ob/ob mice received once daily injections of saline vehicle or (Pro³)GIP (25 nmoles/kg/day) over a 60-day period. Non-fasting plasma glucose levels were significantly reduced in (Pro³)GIP-treated mice compared to controls from day 14 onwards (P<0.05 to P<0.001), concomitantly glycated haemoglobin levels were significantly (P<0.01) decreased in these animals on day 60. In addition, non-fasting plasma insulin was generally lower in (Pro³)GIP treated mice and on day 44 was significantly (P<0.05) less than controls. Sixty-day GIP-R ablation also significantly lowered overall plasma glucose response to feeding (1.7-fold; P<0.05) and an intraperitoneal glucose load (1.9-fold; P<0.001). These changes were associated with significantly enhanced (1.5-fold; P<0.05) insulin sensitivity, reduced pancreatic insulin content (1.5-fold; P<0.01) and significantly decreased levels of circulating triglycerides (P<0.05) and LDL-C(P<0.05). Body weight was decreased in (Pro³)GIP treated ob/ob mice by 17%, but this effect did not achieve statistical significance. However, in normal mice fed high fat or cafeteria diets for 90 days, (Pro³)GIP prevented diet-induced obesity with an up to 20% decrease in body weight (P<0.01). Peptide treatment also blocked the associated deterioration of metabolic control as indicated by a greater than 1% decrease in glycated hemoglobin (P<0.05). The present results emphasize the potential of (Pro³)GIP and GIP receptor blockade for alleviation of obesity and diabetes and the associated abnormalities in insulin resistance, beta cell function and blood lipid profile. This shows that peptide analogues of GIP can be used as a new and effective therapeutic approach for the prevention and treatment of obesity, metabolic syndrome and type 2 diabetes.

The peptide analogue (Pro³)GIP, a specific and potent antagonist of the GIP-R (Gault, V. A., O'Harte, F. P. N, 2002, Biochem. Biophys. Res. Commun. 290:1420-1426), was utilized to assess the effect of extended GIP-R ablation on the metabolic abnormalities of obesity-related diabetes in ob/ob mice. GIP is known to be the major physiological incretin (Gault, V. A. et al., 2003, Diabetologia 46:222-230). As shown herein, once daily administration of (Pro³)GIP to normal mice for 11 days has been shown to result in the reversible impairment of glucose tolerance associated with decreased insulin sensitivity (Irwin, N. et al., 2004, Biol. Chem. 385:845-852). These observations accord with the basic features of GIP receptor knockout mice (GIP-R^{-/-}) (Miyawaki, K. et al., 1999, Proc. Nat. Acad. Sci. USA 96:14843-14847). However, chemical ablation of the GIP receptor appears to have more profound consequences than transgenic knockout, presumably reflecting an adaptive response enabled by lifelong, as opposed to 11 day, deficit in GIP action (Irwin, N. et al, 2004, Biol. Chem. 385:845-852).

Contrary to expectations from studies in normal mice, it is shown herein that short-term blockade of the GIP receptor in adult ob/ob mice by daily (Pro³)GIP administration for 11 days improved many of the characteristic features of type 2 diabetes (Gault, V. A. et al., 2005, Diabetes 54:2436-2446). Administration of (Pro³)GIP daily for 60 days in young (5-7 weeks old) ob/ob mice results in even more marked improvements in both obesity and diabetes status. In particular, glucose and glycated hemoglobin levels remain in the range encountered in normal mice and there is a strong trend for decreased body weight in (Pro³)GIP treated mice. The latter observation is indicative of decreased fat stores and anti-obesity action. Differences in absolute body weight between the two groups of ob/ob mice did not reach significance, but this would be expected by either use of greater numbers of animals, extension of the experimental period or consumption of an energy rich (high fat) diet rather than normal rodent chow.

Numerous beneficial effects of (Pro³)GIP treatment on metabolic parameters were seen. These included decreased fasting and basal hyperglycemia, lowered glycated hemoglobin, improved glucose tolerance and a significantly diminished glycaemic excursion following feeding. Notably, basal and glucose-stimulated plasma insulin concentrations were decreased, suggesting that insulin sensitivity must have improved significantly following (Pro³)GIP in order to restrain the hyperglycemia. Indeed, insulin sensitivity tests conducted after 60 days of (Pro³)GIP administration revealed a significant increase in the glucose-lowering action of exogenous insulin. These combined actions kept glucose and glycated hemoglobin levels of (Pro³)GIP treated ob/ob mice in the normal range and prevented the characteristic progressive elevation of non-fasting glucose observed in ob/ob controls.

Considering the postulated role of GIP on triglyceride levels (Green, B. D. et al., 2004, Curr. Pharm. Des. 10:3651-3662), its location and timing of release, known effects on lipoprotein lipase activity and knowledge that elevated GIP concentrations are present in hypertriglyceridaemic subjects, an effect of (Pro³)GIP on circulating triglycerides seems likely. In animal studies, exogenous GIP has been shown to promote chylomicron triglyceride clearance and lower postprandial circulating triglyceride levels (Yip, R. G., Wolfe, M. M., 2600, Life Sci. 66:91-103). However, as was the case in terms of insulin sensitivity and glucose tolerance, prolonged (Pro³)GIP treatment resulted in unanticipated improvements in lipid status in ob/ob mice. Circulating triglyceride concentrations were significantly lowered in (Pro³)GIP treated mice, and while total cholesterol levels were unaltered when compared to controls, (Pro³)GIP treated mice displayed significantly lowered LDL-C levels. These observations indicate a physiological effect of GIP on lipid metabolism and indicate that GIP receptor blockade represents a new effective approach to improving the plasma lipid profile and affording protection from heart and vascular disease.

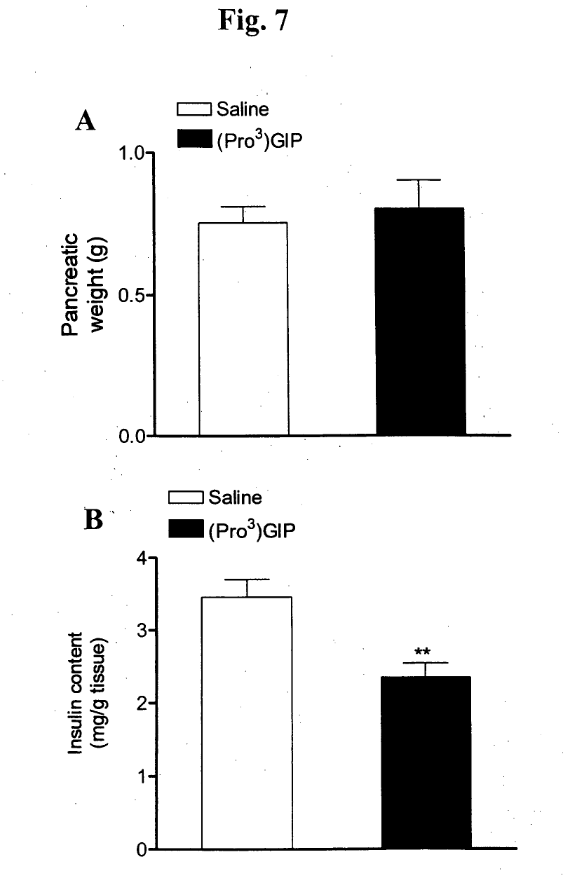

A series of experiments was performed to evaluate the effects of daily administration of (Pro³)GIP for up to 90 days in normal mice fed a high fat diet, a cafeteria diet rich in fat, a high carbohydrate diet or standard laboratory chow. The results are shown in FIG. 7. As expected, mice receiving the two types diet with high fat content exhibited rapid and sustained body weight gain typical of these models of diet-induced obesity. Determination of glycated hemoglobin at 90 days revealed that this was associated with significant and protracted impairment of blood glucose control, likely due to insulin resistance. Administration of (Pro³)GIP to mice receiving high carbohydrate and standard laboratory diet did not affect weight gain nor glycated hemoglobin. However, (Pro³)GIP substantially decreased diet-induced obesity in the other two groups and prevented disturbances in blood glucose control as indicated by normal glycated hemoglobin levels. These observations accord with the antidiabetic actions of (Pro³)GIP in ob/ob mice and indicate clearly the potential of GIP receptor blockade to counter development of obesity.

Additional experiments were conducted to evaluate the antagonistic activities of additional Glu³-substituted peptide analogues. As shown in Examples 8-13, below, the biological activities of analogues (Ala³)GIP, (Lys³)GIP, (Phe³)GIP, (Trp³)GIP, (Tyr³)GIP, (Hyp³)GIP and (Pro³)GIP were studied, along with (Pro³)LysPAL¹⁶GIP and (Hyp³)LysPAL¹⁶GIP. Although not as potent as (Pro³)GIP or (Hyp³)GIP, the peptide analogues (Ala³)GIP, (Phe³)GIP and (Tyr³)GIP also has antagonistic properties. The peptide analogues (Lys³)GIP and (Trp³)GIP had mostly neutral effects. The peptide analogues (Pro³)GIP, (Hyp³)GIP, (Pro³)LysPAL¹⁶GIP and (Hyp³)LysPAL¹⁶ GIP were the most potent antagonists and were the most resistant to breakdown by DPP IV.

The peptide analogue (Pro³)GIP was the most potent antagonist of the group, both in vitro and in vivo. Addition of a LysPAL¹⁶ group had a neutral effect. The peptide analogue (Hyp³)GIP was nearly as potent as (Pro³)GIP, and the addition of a LysPAL¹⁶ group also had a neutral effect.

In both genetic and diet-induced models of obesity-diabetes, long-term administration of the GIP-R antagonist, (Pro³)GIP, counters the development and progression of diabetes, glucose tolerance, insulin resistance and abnormalities of islet structure and function. Lipid status was also significantly improved by (Pro³)GIP treatment, indicating additional benefit in reducing risk from cardiovascular events. Further refinement of the approach concerns the design of longer acting forms of GIP blocker that may include PEGylation of the molecule, fusion with specific protein found in serum or introduction linker groups into the peptide to facilitate protein binding in vivo. These data indicate that GIP receptor antagonism and GIP receptor antagonists, such as (Pro³)GIP, provide a novel, safe and effective means to treat obesity, metabolic syndrome and type 2 diabetes either alone or as combination therapy with dietary manipulation or other antiobesity or antidiabetic drugs.

### Bioavailability and Half-Life of Peptide Analogues

The fact that (Pro³)GIP can be used to chemically ablate the GIP receptor in ob/ob mice and mice with diet-induced obesity indicates the usefulness of the peptide analogue, including forms of the GIP analogues that are N-terminally protected by PEG.

PEG (polyethylene glycol) is a non-antigenic, water-soluble, biocompatible, inert polymer that significantly prolongs the circulatory half-life of a protein (Abuchowski, A. et al., 1984, Cancer Biochem. Biophys. 7:175-186; Hershfield, M. S. et al., 1987, N. Engl. J. Medicine 316:589-596; Meyers, F. J. et al., 1991, Clin. Pharmacol. Ther. 49:307-313), allowing the protein to be effective over a longer time. Covalent attachment of PEG (“PEGylation”) to a protein increases the protein's effective size and reduces its rate of clearance rate from the body.

PEGylation can also result in reduced antigenicity and immunogenicity, improved solubility, resistance to proteolysis, improved bioavailability, reduced toxicity, improved stability, and easier formulation of peptides. Polyethylene glycol also does not aggregate, degrade or denature. Polyethylene glycol conjugates are thus stable and convenient for use in diagnostic assays.

PEGs are commercially available in several sizes, allowing the circulating half-lives of PEG-modified proteins to be tailored for individual indications through use of different size PEGs.

One method for PEGylating proteins is to covalently attach PEG to cysteine residues using cysteine-reactive PEGs. A number of highly specific, cysteine-reactive PEGs with different reactive groups (e.g., maleimide, vinylsulfone) and different size PEGs (2-20 kDa) are commercially available (e.g., from Shearwater, Polymers, Inc., Huntsville, Ala., USA). At neutral pH, these PEG reagents selectively attach to “free” cysteine residues, i.e., cysteine residues not involved in disulfide bonds. The conjugates are hydrolytically stable. Use of cysteine-reactive PEGs allows the development of homogeneous PEG-protein conjugates of defined structure.

Native GIP(1-42) has no cysteines, however, considerable progress has been made in recent years in determining the structures of commercially important protein therapeutics and understanding how they interact with their protein targets, e.g., cell-surface receptors, proteases, etc. This structural information can be used to design PEG-protein conjugates using cysteine-reactive PEGs. Cysteine residues in most proteins participate in disulfide bonds and are nQt available for PEGylation using cysteine-reactive PEGs. Through in vitro mutagenesis using recombinant DNA techniques, additional cysteine residues can be introduced anywhere into the protein. The added cysteines can be introduced at the beginning of the protein, at the end of the protein, between two amino acids in the protein sequence or, preferably, substituted for an existing amino acid in the protein sequence. The newly added “free” cysteines can serve as sites for the specific attachment of a PEG molecule using cysteine-reactive PEGs. The added cysteine must be exposed on the protein's surface and accessible for PEGylation for this method to be successful. If the site used to introduce an added cysteine site is non-essential for biological activity, then the PEGylated protein will display essentially wild type (normal) in vitro bioactivity. When PEGylating proteins with cysteine-reactive PEGs, one should first identify the surface exposed, non-essential regions in the target protein where cysteine residues can be added or substituted for existing amino acids without loss of bioactivity.

Other approaches to extending the bioavailability of N-terminal analogues of GIP can also be employed by exploiting their binding to larger long lived proteins (Dennis, M. S. et al., 2002, J. Biol. Chem. 277:35035-35043). These approaches include genetic fusion with albumin or other plasma proteins, where the gene for GIP is fused with that of the larger protein (Osborn, B. L. et al., 2002, Eur. J. Pharmacol. 456:149-158).

Alternatively, drug affinity complex (DAC) technology (Holmes, D. L. et al., 2000, Bioconj. Chem. 11:439-444) can be employed whereby a short covalent chemical linker is introduced into the C-terminus of the N-terminally modified GIP molecule. This then interacts with a specific cysteine residue of circulating albumin to promote binding of the two molecules.

The binding of modified GIP to albumin or other large proteins can also be achieved by covalent linkage of the peptide to an antibody fragment that reacts with the longer lived protein in vivo.

Small molecular non-peptidergic activators or stimulators of the GIP receptor and associated signaling pathway can also be used. Thus, knowledge of the activity and 3-dimensional NMR structure of the bioactive domain of GIP (Alna, I. et al., 2004, Biochem. Biophys. Res. Comm. 325:281-286; Green, B. D. et al., 2004, Curr. Pharm. Design 10:3651-3662) would facilitate computer-aided ligand- and receptor-based drug design (Honma, T., 2003, Medic. Res. Rev. 23:606-632). Alternatively, screening of in silico databases containing non-peptide small molecules may be useful to identify prospective candidate GIP receptor mimetics and antagonists for biological testing (Alvarez, J. C., 2004, Curr. Opin. Chem. Biol. 8:365-370; Yoshimora, A. et al., 2005, Apoptosis 10:323-329).

These and similar alterations to extend the bioavailability and half-life of the peptide analogues are intended to be included in the invention.

The peptide analogues of the present invention have use in decreasing or preventing obesity and in preventing weight gain and promoting weight loss. One or more of the peptide analogues can be used in a pharmaceutical composition, which can include a pharmaceutically acceptable carrier. Such a composition may also contain (in addition to the analogue and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration.

Administration of the peptide analogues disclosed herein in the pharmaceutical composition or to practice the use of the present invention can be carried out in a variety of conventional ways, such as by oral ingestion, inhalation, topical application or cutaneous, subcutaneous, intraperitoneal, parenteral or intravenous injection. Administration can be internal or external; or local, topical or systemic.

The compositions containing a peptide analogue of this invention can be administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent, i.e., carrier or vehicle.

Formulations suitable for parenteral administration include aqueous and non-aqueous sterile injection solutions which may contain anti-oxidants; buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

When a therapeutically effective amount of the composition of the present invention is administered orally, the composition of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the composition of the present invention, and preferably from about 1 to 50% of the composition of the present invention.

When a therapeutically effective amount of the composition of the present invention is administered by intravenous, cutaneous or subcutaneous injection, the composition of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to the composition of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

Use of timed release or sustained release delivery systems are also included. A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The therapeutic compositions can include pharmaceutically acceptable salts of the components therein, e.g., which may be derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al., describe pharmaceutically acceptable salts in detail in J. Pharmaceutical Sciences, 1977, 66:1 et seq., which is incorporated herein by reference in its entirety. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. The salts may be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptonoate, hexanoate, fumarate, hydrochloride, hydrobromide, hydroiodide, 2-hydroxymethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid:

As used herein, the terms “pharmaceutically acceptable”, “physiologically tolerable” and grammatical variations thereof as they refer to compositions, carriers, diluents and reagents, are used interchangeably and represent that the materials are capable of administration to or upon a mammal with a minimum of undesirable physiological effects such as nausea, dizziness, gastric upset and the like. The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The amount of peptide analogue of the present invention in the pharmaceutical composition of the present invention will depend upon the nature and severity of the condition being treated, on the nature of prior treatments which the patient has undergone, and on a variety of other factors, including the type of injury, the age, weight, sex, medical condition of the individual. Ultimately, the attending physician will decide the amount of the analogue with which to treat each individual patient. Initially, the attending physician will administer low doses of peptide analogue and observe the patient's response. Larger doses of peptide analogue may be administered until the optimal therapeutic effect is obtained for the patient, and at that point the dosage is not increased further.

Additional guidance on methods of determining dosages can be found in standard references, for example, Spilker, Guide to Clinical Studies and Developing Protocols, Raven Press Books, Ltd., New York, 1984, pp. 7-13 and 54-60; Spilker, Guide to Clinical Trials, Raven Press, Ltd., New York, 1991, pp. 93-101; Craig et al., Modern Pharmacology, 2d ed., Little Brown and Co., Boston, 1986, pp. 127-133; Speight, Avery's Drug Treatment: Principles and Practices of Clinical Pharmacology and Therapeutics, 3d ed., Williams and Wilkins, Baltimore, 1987, pp. 50-56; Tallarida et al., Principles in General Pharmacology, Springer-Verlag, New York, 1998, pp. 18-20; and Olson, Clinical Pharmacology Made Ridiculously Simple, MedMaster, Inc., Miami, 1993, pp. 1-5.

### Combination Therapies

Any of the peptide analogues as disclosed herein can be combined with other antidiabetic treatments. As used herein, “antidiabetic treatments” include agents used to treat or ameliorate diabetic symptoms, and agents having an antidiabetic effect. Such agents can include pharmaceutical agents such as, but not limited to, chemical, biochemical, peptide, peptidomimetic agents. Peptide agents can include one or more of the peptide analogues as disclosed herein, or other GIP receptor antagonists. The peptide analogues can also be combined with other treatment regimens such as dietary regimens. The combination of a stable GIP receptor antagonist with another antagonistic antidiabetic agent can be an effective means of treating or preventing obesity or preventing weight gain or promoting weight loss. Various antidiabetic drugs are discussed below.

### Biguanides

Biguanides decrease glucose production by the liver. Metformin is a biguanide, and is marketed under the names “Glucophage” (metformin HCl tablets; Bristol-Myers Squibb Company) and “Glucophage XR” (metformin HCl extended release tablets; Bristol-Myers Squibb Company). Metformin also lowers-total cholesterol; low density lipoproteins and triglycerides, and raises beneficial high density lipoproteins. Metformin is not generally used in patients with impaired liver or renal function, congestive heart failure, unstable heart disease, hypoxic lung disease, or advanced age.

### Sulphonylureas

Sulphonylurea medications work by increasing the amount of insulin made by the pancreas, and may be used for those patients who cannot take metformin. Possible side-effects include weight gain and hypoglycemia. Sulphonylureas include “first generation” sulfonylureas, which were marketed before 1984 (acetohexamide (Dymelor; Eli Lilly and Company), chlorpropamide (Diabinese; Pfizer Inc.), tolbutamide (Orinase; Pharmacia & Upjohn Inc.), tolazamide (Tolinase; Pharmacia & Upjohn Inc.)), and “second generation” sulfonylureas, which have been marketed since 1984 (glimepiride (Amaryl; Sanofi-Aventis S.A.), gliclazide (Diamicron; Servier), glipizide (Glucotrol, Glucotrol XL; Pfizer Inc.), glyburide, or glibenclamide (Diabeta; Aventis S.A.; Glynase PresTab, Micronase; Pharmacia & Upjohn Inc.)).

### Thiazolidinediones

Thiazolidinediones, also called-glitazones, lower blood glucose by increasing insulin sensitivity, and can be taken in addition to metformin or a sulphonylurea. Thiazolidinediones include pioglitazone (e.g., pioglitazone hydrochloride (Actos; Takeda Chemicals Industries Ltd., Eli Lilly)), rosiglitazone (rosiglitazone maleate (Avandia; GlaxoSmithKline)), and troglitazone (Rezulin; Parke-Davis/Warner-Lambert).

### Meglitinides

Meglitinides stimulate insulin secretion of pancreatic beta cells, but are of a shorter duration than the sulphonyureas. Possible side-effects include weight gain and hypoglycemia They can be administered alone or in combination with metformin. They include nateglinide (Starlix; Novartis Pharma AG) and repaglinide (Prandin, NovoNorm, or GlucoNorm; Novo Nordisk A/S).

### Alpha-Glucosidase Inhibitors

Alpha-glucosidase inhibitors delay the digestion of sugars and starches by delaying the absorption of carbohydrates from the gut, thereby reducing peaks of blood glucose which may occur after meals. Such inhibitors include acarbose (Precose, Prandase; Bayer North America), miglitol (Glyset, Diastabol; Pharmacia & Upjohn Inc., Sanofi).

### Incretin Mimetics

These drugs enhance glucose-dependent insulin secretion by pancreatic beta-cells, suppress inappropriately elevated glucagon secretion, and slow gastric emptying. Exenatide (Byetta; Amylin Pharmaceuticals Inc.) is an incretin mimetic. It lowers blood glucose levels by increasing insulin secretion. It does this only in the presence of elevated blood glucose levels, and so tends not to increase the risk of hypoglycemia. Hypoglycemia can still occur if it is combined with a sulfonylurea, however. It is used to treat type 2 diabetes.

### Amylinomimetics

Pramlintide (e.g., pramlintide acetate; Symlin; Amylin Pharmaceuticals, Inc.) is a synthetic analogue of the hormone amylin, which is produced by pancreatic beta cells. Amylin, insulin and glucagon work together to maintain normal blood glucose levels. Pramlintide has been approved for patients with type 1 and type 2 diabetes. Pramlintide cannot be combined with insulin and must be injected separately.

### Other Antidiabetic Agents

Other agents, such as guar gum, can be used to slow intestinal glucose absorption. Agents to increase glucose excretion are also under development.

### Combination Drugs

Combination drugs are also available, such as those which combine metformin with another oral medication (e.g., glyburide and metformin HCl (Glucovance; Bristol-Myers Squibb Company), rosiglitazone maleate and metformin HCl (Avandamet; GlaxoSmithKline), and glipizide and metformin HCl (Metaglip; Bristol-Myers Squibb Company)).

### Obesity Medications

The peptide analogues disclosed herein can also be combined with other anti-obesity, treatments, such as, but not limited to, sibutramine HCl monohydrate C-IV (Meridia®), orlisat (Xenical®), or other treatments such as diets and dietary formulations. Treatments in the development stage include, but are not limited to, rimonabant (Accomplia), cannabinoid antagonists, lipase inhibitors, dual serotonin and norepinephrin reuptake inhibitors, beta-3 agrenergic agonists, cholecystokinin agonists, ciliary neurotrophic factor (CNTF) agonists, leptin antagonists and lipid metabolism modulators.

Antagonism of cannabinoid receptor CB1 is shown to reduce food intake and increase energy expenditure. Dual serotonin (5-HT) plus norepinephrine reuptake inhibitors (SNRIs) (e.g., Meridia®; Abbott) reduce food intake by either a central mechanism reducing food intake or a peripheral mechanism increasing thermogenesis. Beta-3 adrenergic agonists (e.g., SR58611; Sanofi-Aventis) regulate energy metabolism and thermogenesis, particularly in response to norepinephrine. Cholecystokinin agonists (e.g., GI 181771, GlaxoSmithdline) slow gastric emptying and induce release of pancreatic enzymes and gallbladder contraction, and so influence feeding behaviour. Ciliary neurotropic factor (CNTF) agonists include Axokine (Regeneron), which is a cytokine and analogue of CNTF with strong neuroprotective effects and similarities to leptin. The leptin receptor and CNTFR-alpha have overlapping distribution and possible common action. CNTR may be an alternative to treating patients with leptin so as to activate the same or a similar mechanism. Lipase inhibitors such as tetrahydrolipstatin (e.g., Orlistat), inhibit gastric and pancreatic lipases in the lumen of the gastrointestinal tract so as to decrease systemic absorption of dietary fat.

Leptin is a naturally occurring hormone secreted by fat cells that may suppress appetite and enhance metabolism. It is a member of the interleukin-6 cytokine family, is found in multiple tissues and is secreted by white adipose tissue. Because of its action in suppressing appetite, leptin agonists are thought to be potential treatments for overeating. Lipid metabolism modulators may also become treatments for obesity, such as the peptide variant of hGH 177-191 (e.g., AOD9604; Metabolic Pharmaceuticals), which is a region of growth hormone molecule hGH 177-191 which may be responsible for its specific effect on fat without effect on growth or insulin resistance. It may act as replacement therapy for hGH-deficient state preceding age-onset obesity.

### Other Peptide Sequences

In the uses disclosed herein, it is preferable that analogues of human GIP are used. However, the GIP protein sequences from a number of animals are very similar to that of human, and these can also be used in the treatment methods disclosed herein. As used herein, the term a “peptide analogue of GIP” is intended to include other mammalian GIP polypeptide sequences which are similar to the human sequence and which can be used in the invention. The sequences for human (Moody et al. 1984 FEBS Lett. 172: 142-148), pig (Brown & Dryburgh 1971 Can. J. Biochem. 49: 867-872), cow (Carlquist et al. 1984 Eur. J. Biochem. 145: 573-577), hamster (Yasuda et al. 1994 Biochem. Biophys. Res. Commun. 205: 1556-1562), rat (Higashimoto et al. 1992 Biochim. Biophys. Acta 1132: 72-74) and mouse (Schieldrop et al. 1996 Biochim. Biophys. Acta 1308: 111-113) are provided below. The porcine GIP sequence differs from human at residues 18 and 34, while the bovine GIP sequence also differs at residue 37, etc. For all of the animal protein sequences, variations from the human primary sequence are capitalized and underlined.

(SEQ ID NO:1)

| | |
|---|---|
| Human: | yaegtfisdysiamdkihqqdfvnwllaqkgkkndwkhnitq |

(SEQ ID NO:2)

| | |
|---|---|
| Pig: | yaegtfisdysiamdkiRqqdfvnwllaqkgkkSdwkhnitq |

(SEQ ID NO:3)

| | |
|---|---|
| Cow: | yaegtfisdysiamdkiRqqdfvnwllaqkgkkSdwIhnitq |

(SEQ ID NO:4)

| | |
|---|---|
| Hamster: | yaegtfisdysiamdkiRqqdfvnwllaqkgkkndwkhnitq |

(SEQ ID NO:5)

| | |
|---|---|
| Rat: | yaegtfisdysiamdkiRqqdfvnwllaqkgkkndwkhnLtq |

(SEQ ID NO:6)

| | |
|---|---|
| Mouse: | yaegtfisdysiamdkiRqqdfvnwllaqRgkkSdwkhnitq |

Species homologues of the disclosed proteins are also provided by the present invention. As used herein, a “species homologue” is a protein or polynucleotide with a different species of origin from that of a given protein or polynucleotide, but with significant sequence similarity to the given protein or polynucleotide. Preferably, polypeptide species homologues have at least 90% sequence identity, more preferably at least 95% identity most preferably at least 97% or 100% identity with the given polypeptide, where sequence identity is determined by comparing the amino acid sequences of the proteins when aligned so as to maximize overlap and identity while minimizing sequence gaps. Species homologues may be isolated and identified by making suitable probes or primers from the sequences provided herein and screening a suitable nucleic acid source from the desired species. Preferably, species homologues are those isolated from mammalian species. Most preferably, species homologues are those isolated from certain mammalian species such as, for example, primates, swine, cow, sheep, goat, hamster, rat, mouse, horse, or other species possessing GIP proteins of significant homology to that of human.

The invention also encompasses allelic variants of the disclosed GIP proteins; that is, naturally-occurring alternative forms of the GIP polypeptide which are identical or have significantly similar sequences to those disclosed herein. Preferably, allelic variants have at least 90% sequence identity, more preferably at least 95% identity most preferably at least 97% or 100% identity with the given polypeptide, where sequence identity is determined by comparing the amino acid sequences of the proteins when aligned so as to maximize overlap and identity while minimizing sequence gaps. Allelic variants may be isolated and identified by making suitable probes or primers from the sequences provided herein and screening a suitable nucleic acid source from individuals of the appropriate species.

The therapeutic compositions are also presently valuable for veterinary applications. Particularly domestic animals and thoroughbred horses, in addition to humans, are desired patients for such treatment with proteins of the present invention.

### EXAMPLES

### Example 1

### Experimental Methods

### Synthesis, Purification and Characterization of (Pro³)GIP

(Pro³)GIP was sequentially synthesized on an Applied Biosystems automated peptide synthesizer (Model 432 A) as reported previously (Gault, V. A., O'Harte, F. P .M., 2002, Biochem. Biophys. Res. Commun. 290:1420-1426). (Pro³)GIP was purified by reversed-phase HPLC on a Waters Millenium 2010 chromatography system (Software version 2.1.5) and subsequently characterized using electrospray ionization mass spectrometry (ESI-MS) as described elsewhere (Gault, V. A., O'Harte, F. P. M., 2002, Biochem. Biophys. Res. Commun. 290:1420-1426).

### Animals

Young obese diabetic (ob/ob) mice derived from the colony maintained at Aston University, UK (Bailey, C. J. et al., 1982, Int. J. Obes. 6:11-21) were used at 5-7 weeks of age. Normal lean control mice from the same colony were used in comparative experiments (See Example 6, below). Animals were age-matched, divided into groups and housed individually in an air-conditioned room at 22±2° C. with a 12 hours light:12 hours dark cycle (08:00-20:00 hours). Drinking water and a standard rodent maintenance diet (Trouw Nutrition, Cheshire, UK) were freely available. In a separate experiment, Swiss Tylers Original (TO) mice purchased from Harlan Ltd. (Bicester, UK) were used at 5-7 weeks of age. Animals were housed as for ob/ob mice and given drinking water ad libitum. Normal TO mice were allowed free access to fed high fat diet (45% fat, 20% protein, 35% carbohydrate, 26.15 MJ/kg), high carbohydrate diet (10% fat, 20% protein, 70% carbohydrate, 18.80 MJ/kg) (Special Diets Service, Witham, Essex, UK), cafeteria diet (corresponding to approximately 33% fat, 19% protein, 48% carbohydrate, 16.39 MJ/kg) or normal rodent maintenance diet (10% fat, 30% protein, 60% carbohydrate; 14.2 MJ/kg, Trouw Nutrition, Cheshire, UK). The cafeteria diet comprised 6 daily rotations of 2 palatable food items per day (2 from: tuna, peanut butter, crisps, chocolate biscuits, madera cake, chocolate, luncheon meat, sausages, corned beef, cheese, marzipan). All animal experiments were carried out in accordance with the UK Animals (Scientific Procedures) Act 1986.

### Experimental Protocols

ob/ob mice received, over an 60-day period, once daily i.p. injections (17:00 h) of either saline vehicle (0.9% (w/v), NaCl) or (Pro³)GIP (25 nmol/kg body wt). Food intake and body weight were recorded daily while plasma glucose and insulin concentrations were monitored at intervals of 3-6 days. Whole blood for the measurement of glycated hemoglobin was taken on day 60. In addition, plasma samples for measurement of cholesterol, circulating triglycerides, HDL-cholesterol (HDL-C) and LDL-cholesterol (LDL-C) were taken on day 60. Intraperitoneal glucose tolerance (18 mmol/kg body wt), metabolic response to native GIP (25 nmol/kg body wt) and insulin sensitivity (50 U/kg body wt) tests were performed on day 60. Mice fasted for 18 hours were used to examine the metabolic response to 15 minutes feeding on day 60. In a separate series, pancreatic tissues were excised at the end of the 60-day treatment period and processed for immunohistochemistry or measurement of insulin following extraction with 5 ml/g of ice-cold acid ethanol (750 ml ethanol, 235 ml water, 15 ml concentrated HCl). Blood samples taken from the cut tip of the tail vein of conscious mice at the times indicated in the Figures were immediately centrifuged using a Beckman microcentrifuge (Beckman instruments, UK) for 30 seconds at 13,000×g. The resulting plasma was then aliquoted into fresh Eppendorf tubes and stored at −20° C. prior to glucose and insulin determinations. Normal control mice from the same genetic background were used for comparative purposes. In the case of TO mice, groups of animals received, over a 90 day period, once daily intraperitoneal (i.p.) injections (17:00 h) of either (Pro³)GIP(1-16) (25 nmol/kg body weight) or saline vehicle (0.9%, w/v, NaCl). Body weights were recorded daily and blood for determination of glycated hemoglobin was collected at end of study period.

### Biochemical Analysis

Plasma glucose was assayed by an automated glucose oxidase procedure (Stevens, J. F., 1971, Clin. Chem. Acta 32:199-201) using a Beckman Glucose Analyzer II (Beckman Instruments, Galway, Ireland). Plasma and pancreatic insulin were assayed by a modified dextran-coated charcoal radioimmunoassay (Flatt, P. R., Bailey, C. J., 1981, Diabetologia 20:573-577). Plasma triglyceride and cholesterol levels were measured using a Hitachi Automatic Analyser 912 (Boehringer Mannheim, Germany). Glycated hemoglobin was determined using a commercially available kit purchased from Chirus Ltd. (Watford, UK).

### Immunocytochemistry

Tissue fixed in 4% paraformaldehyde/PBS and embedded in paraffin was sectioned at 8 μm. After de-waxing, and exposure to insulin antibody, samples were stained and counter-stained as described previously (Gault, V. A. et al., 2005, Diabetes 54:2436-2446). The stained slides were viewed under a microscope (Nikon Eclipse E2000, Diagnostic Instruments Incorporated, Michigan, USA) attached to a JVC camera Model KY-F55B (JVC, London, UK) and analyzed using Kromoscan imaging software (Kinetic Imaging Limited, Faversham, Kent, UK). The average number and diameter of every islet in each section was estimated in a blinded manner using an eyepiece graticule calibrated with a stage micrometer (Graticules Limited, Tonbridge, Kent, UK). The longest and shortest diameters of each islet were determined with the graticule. Half of the sum of these two values was then considered to be the average islet diameter. Approximately 60-70 random sections were examined from the pancreas of each mouse.

### Statistics

Results are expressed as mean±SEM. Data were compared using ANOVA, followed by a Student-Newman-Keuls post hoc test. Area under the curve (AUC) analyzes were calculated using the trapezoidal rule with baseline subtraction. P<0.05 was considered to be statistically significant.

### Example 2

### Effects of (Pro³)GIP on Food Intake, Body Weight, Glycated Hemoglobin and Non-Fasting Plasma Glucose and Insulin Concentrations in Ob/Ob Mice

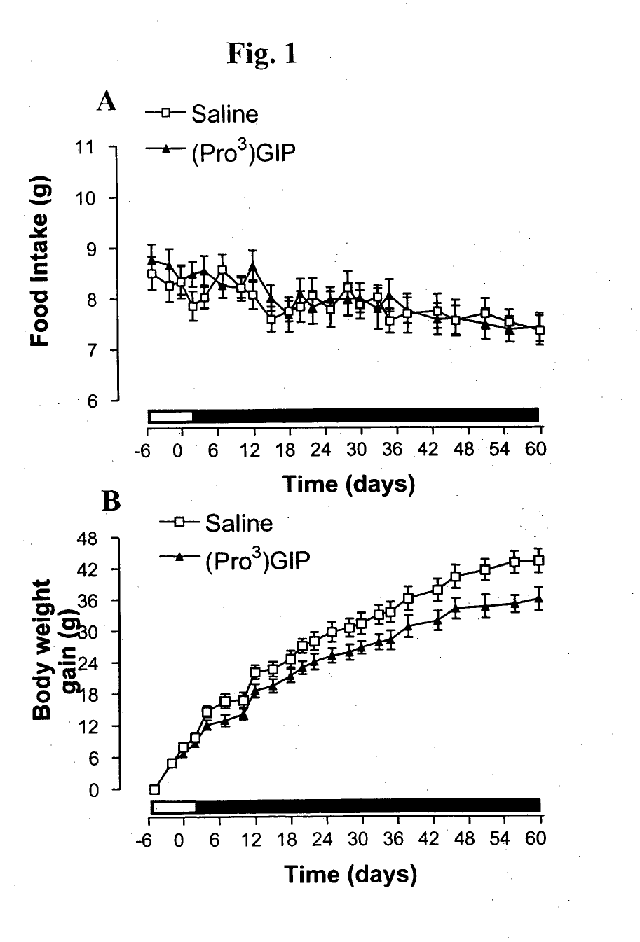

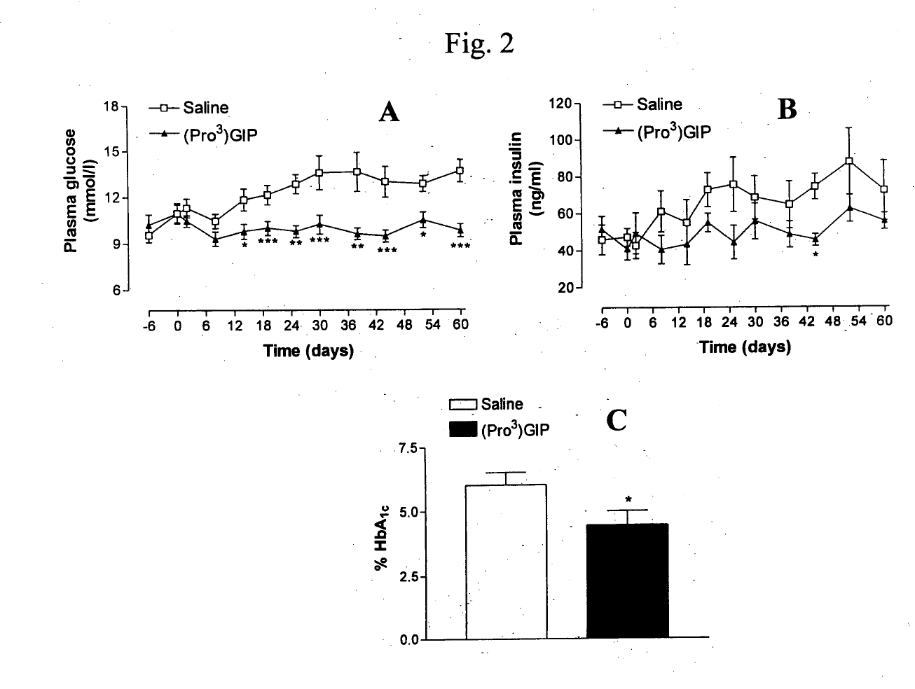

This example examined the effects of daily (Pro³)GIP administration on food intake and body weight of ob/ob mice, and non-fasting plasma glucose, plasma insulin and glycated haemoglobin concentrations of ob/ob mice. The results are shown in FIGS. 1A and 1B, which are a pair of line graphs, and FIGS. 2A-2C, which are a pair of line graphs and a bar graph. Administration of (Pro³)GIP (▴) for 60 days had no effect on food intake (FIG. 1B) relative to control (saline; □). While there was an approximate 17% decrease in body weight, this did not reach significance over the study period, as shown in FIG. 1A. On day 14, plasma glucose had declined to significantly reduced (P<0.05) concentrations in ob/ob mice receiving (Pro³)GIP (▴) (FIG. 2A) and subsequently remained significantly lowered compared to control (□) until day 60 (P<0.05 to P<0.001). Consistent with this pattern, glycated hemoglobin was significantly lower (P<0.05) after 60 days treatment with (Pro³)GIP (black bar) (6.1±0.4%, vs. 4.1±0.1%), relative to control (white bar) (FIG. 2C). Plasma insulin levels had a tendency to be lower in (Pro³)GIP treated mice (▴), and on day 44 were significantly lowered (P<0.05) compared to controls (□) (FIG. 2B). Glucose and glycated hemoglobin levels of age-matched normal control mice (8.8±0.3 mmol/l and 4.8±0.2%, respectively) were not dissimilar to those of ob/ob mice treated with (Pro³)GIP.

### Example 3

### Effects of (Pro³)GIP on Glucose Tolerance and Response to Native GIP in Ob/Ob Mice

This example evaluated the effects of daily (Pro³)GIP administration on glucose tolerance and plasma insulin response to glucose in ob/ob mice and on metabolic response to native GIP, also in ob/ob mice.

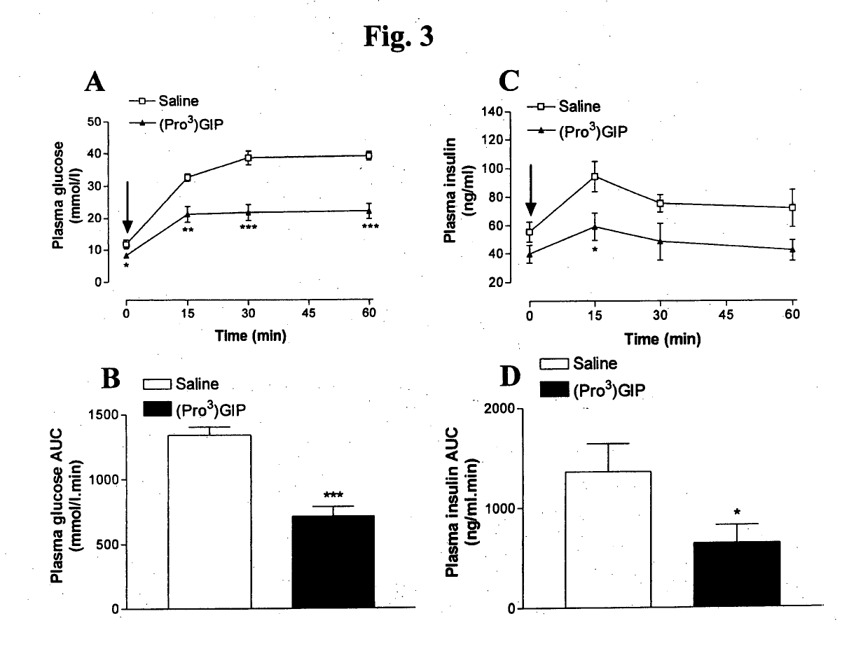

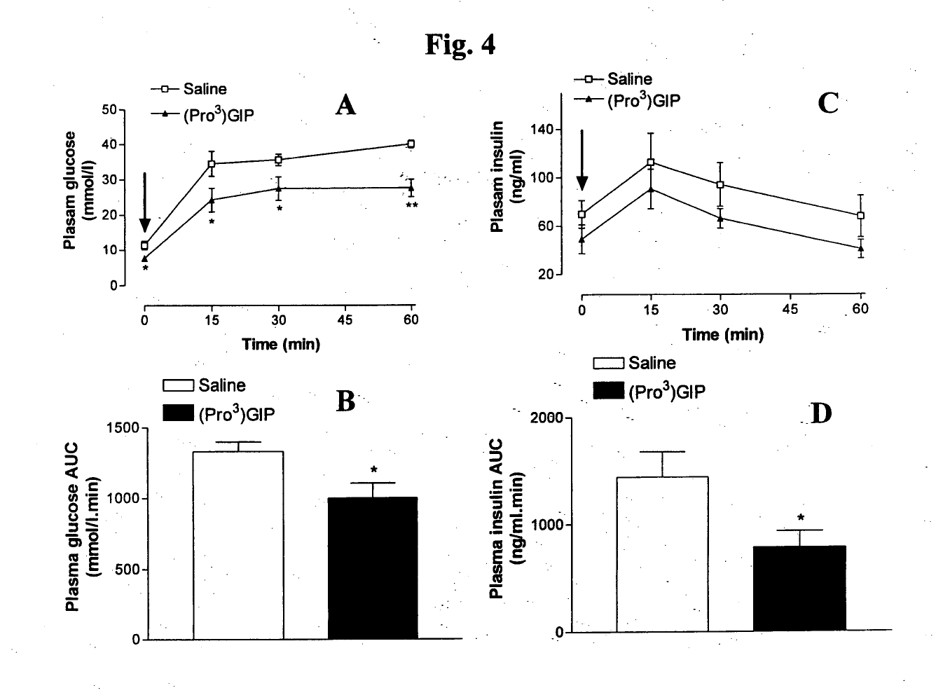

Daily administration of (Pro³)GIP (▴) for 60 days resulted in significantly reduced (P<0.001) plasma glucose concentrations at 0, 15, 30 and 60 minutes following intraperitoneal glucose (FIG. 3A), relative to controls (□). This was corroborated by a significantly (P<0.001) decreased 0-60 minutes AUC value (FIG. 3B) ((Pro³)GIP treatment, black bar; saline control, white bar). Plasma insulin concentrations were also significantly (P<0.05) reduced at 15 minutes following intraperitoneal glucose injection in the (Pro³)GIP treated group (▴) (FIG. 3C), relative to controls (□). AUC, 0-60 minute values were also significantly decreased (P<0.05) ((Pro³)GIP treatment, black bar; saline control, white bar) (FIG. 3D). Interestingly, a similar pattern was observed when 60 day treated ob/ob mice were administered glucose together with native GIP (25 nmoles/kg bw) (FIG. 4). There was a significant decrease (P<0.05) in both the overall glycaemic excursion and insulinotropic response in the 60 day (Pro³)GIP treated mice compared to control following GIP administration. This supports the view that GIP action was effectively antagonized in the (Pro³)GIP treated group.

### Example 4

### Effects of (Pro³)GIP on Metabolic Response to Feeding and Insulin Sensitivity in Ob/Ob Mice

This example looked at the effects of daily (Pro³)GIP administration on glucose and insulin responses to feeding in 18 hour-fasted ob/ob mice, and on insulin sensitivity in ob/ob mice.

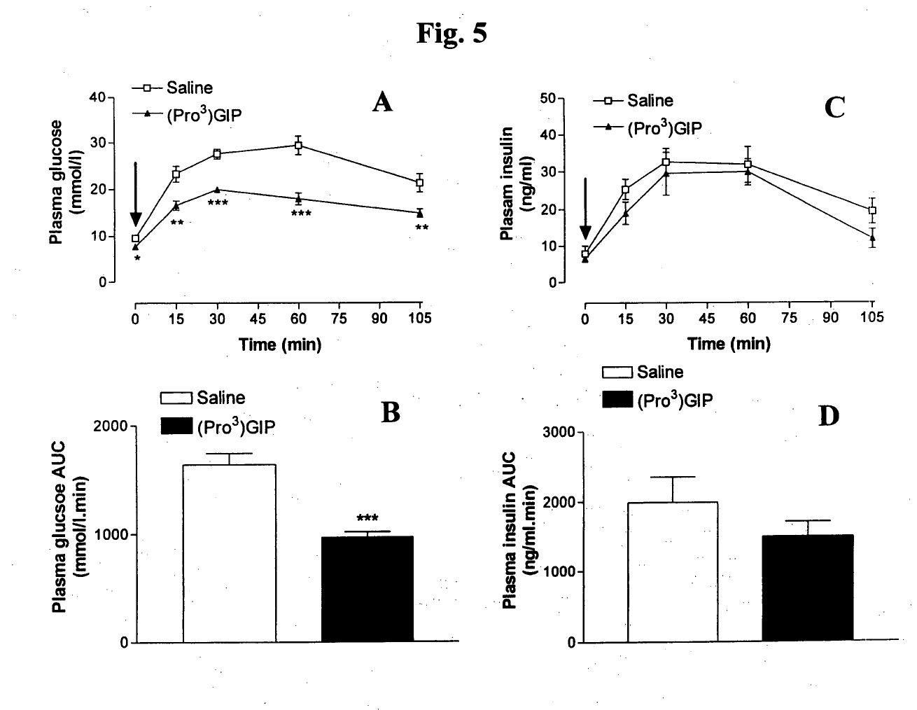

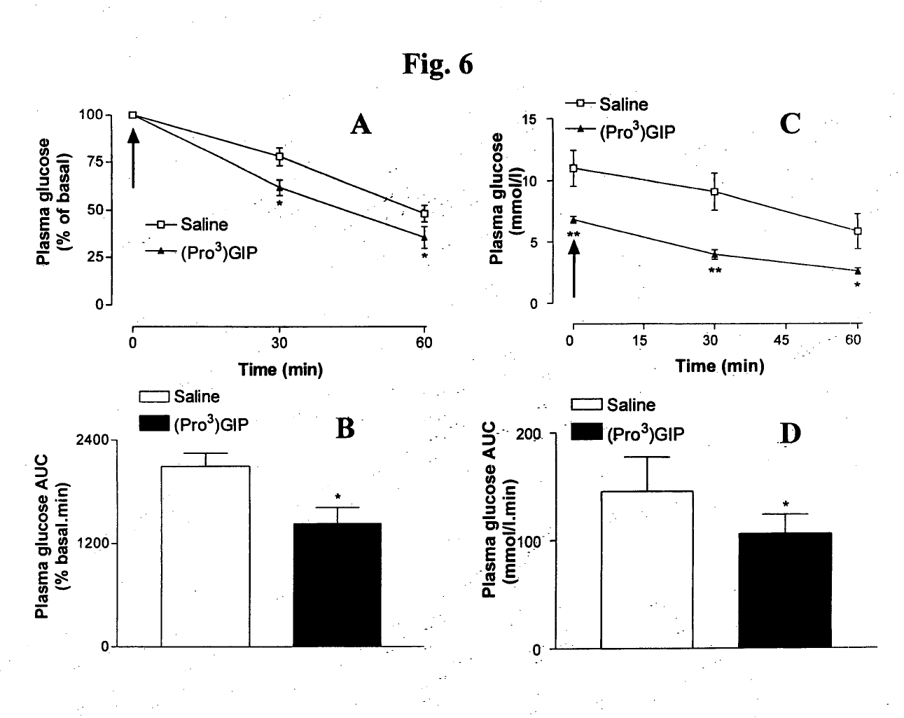

Plasma glucose responses to 15 minute feeding was significantly lowered at 15, 30, 60 and 105 minutes (P<0.05 to P<0.001) in ob/ob mice treated with (Pro³)GIP (▴; black bar) for 60 days (FIGS. 5A and 5B), relative to saline-treated controls (□; white bar). This was translated to a significantly (P<0.05) decreased overall glycaemic excursion in (Pro³)GIP treated ob/ob mice, despite similar food intakes of 0.4-0.6 g/mouse/15 minutes. Surprisingly, plasma insulin levels (FIGS. 5C and 5D) were not significantly different between the two groups ((Pro³)GIP, ▴, black bar; saline controls, □, white bar). As shown in FIGS. 6A-6D, the hypoglycemic action of insulin was significantly (P<0.05) augmented in terms of AUC measures and post injection values in ob/ob mice treated with (Pro³)GIP for 60 days. FIGS. 6A and 6B show plasma glucose and plasma glucose AUC, respectively, as % of basal values, and FIGS. 6C and 6D show plasma glucose and plasma glucose AUC as whole numbers.

### Example 5

### Effects of (Pro³)GIP on Pancreatic Insulin in Ob/Ob Mice

The effects of daily (Pro³)GIP administration on pancreatic weight and insulin content were also examined.

As shown in FIG. 7A, (Pro³)GIP treatment (black bars) had no effect on pancreatic weight, relative to controls (white bars). However, pancreatic insulin content was significantly (P<0.01) decreased in ob/ob mice receiving (Pro³)GIP for 60 days compared to controls (FIG. 7B).

### Example 6

### Effects of (Pro³)GIP on Circulating Triglycerides and Cholesterol in Ob/Ob Mice

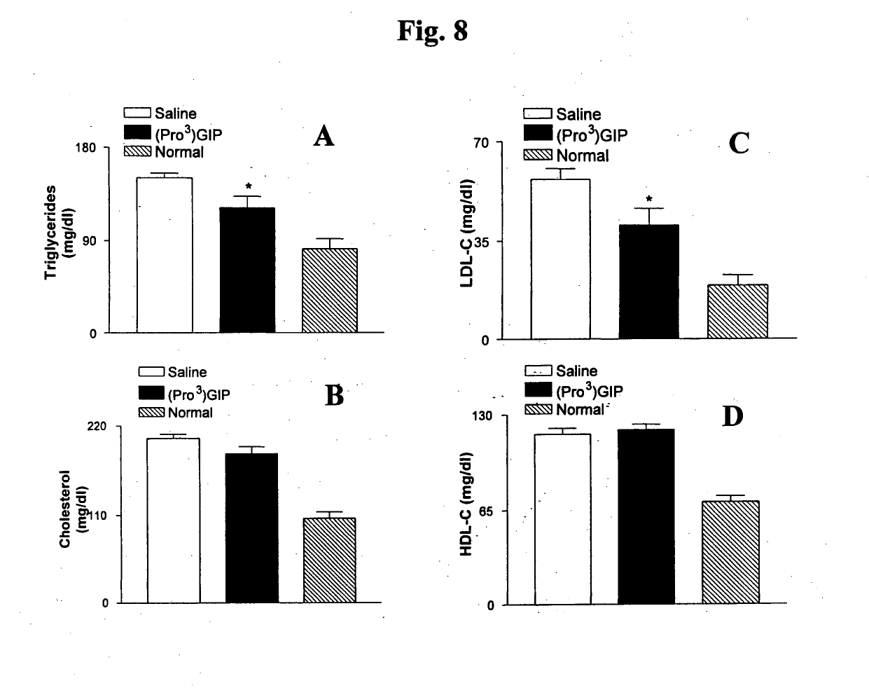

The effects of daily (Pro³)GIP administration on lipid profile in (ob/ob) mice was also studied. The results are shown in FIGS. 8A-8D.

(Pro³)GIP treatment for 60 days significantly (P<0.05) reduced circulating triglyceride concentrations in ob/ob mice (FIG. 8A). Cholesterol levels (FIG. 8B) were unaltered between control and (Pro³)GIP treated mice, however, more detailed assessment of circulating cholesterol revealed significantly reduced (P<0.05) levels of LDL-C (FIG. 8C) in 60 day (Pro³)GIP ob/ob mice. Levels of HDL-C are shown in FIG. 8D. Values for cholesterol, triglycerides, HDL-C and LDL-C from normal lean control mice from the same colony are shown for comparison (“normal”; cross-hatched bars).

### Example 7

### Effects of Daily (Pro³)GIP Administration on Body Weight and Glycated Hemoglobin of Normal (To) Mice Fed High Fat, High Carbohydrate, Cafeteria and Normal Diets

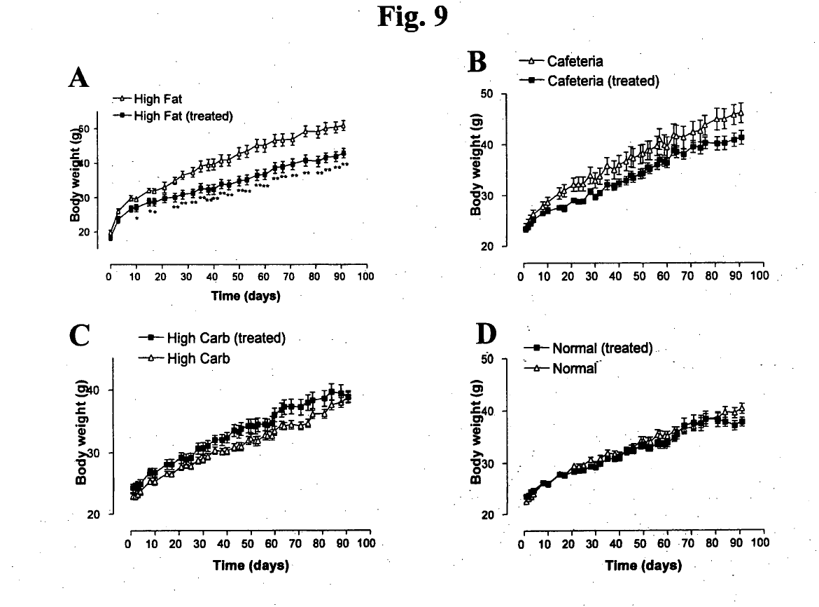

This example shows the effects of daily (Pro³)GIP administration on body weight of normal (TO) mice fed high fat, high carbohydrate, cafeteria and normal diets. The results are shown in FIGS. 9A-9D, which are a set of line graphs.

Normal mice were fed the different diets indicated above ad libitum from 4-5 weeks of age. Treated groups of mice received intraperitoneal injection of ProGIP (25 nmoles/kg body weight) each day. (Pro³)GIP clearly counters body weight gain induced by excessive energy intake in animals receiving high fat diet and to a lesser extent those fed on cafeteria items.

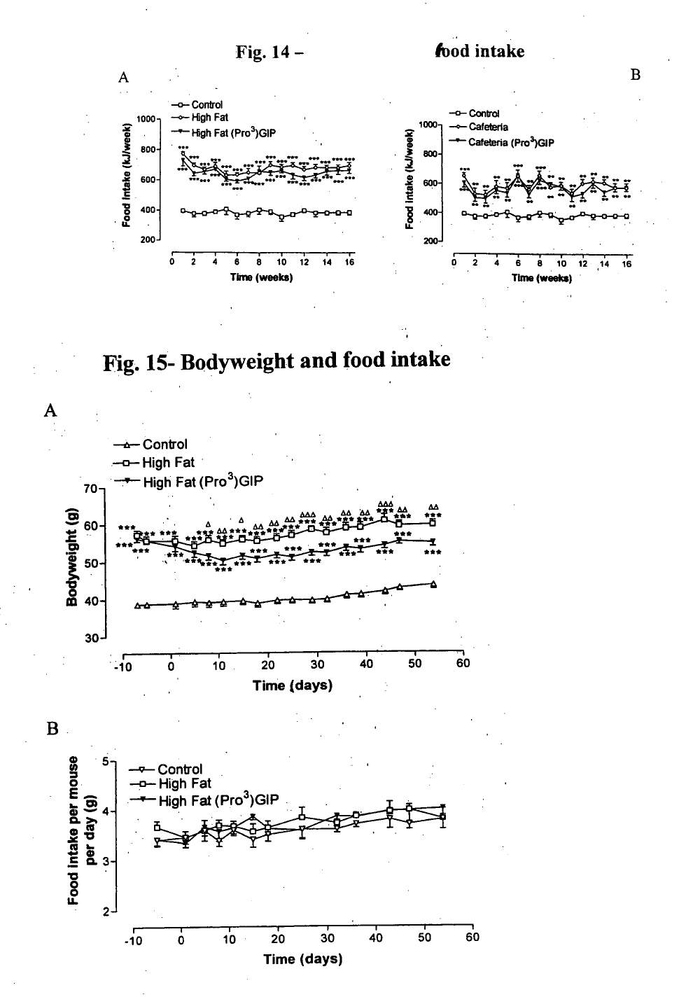

High fat diet (FIG. 9A) and cafeteria diet rich in fat (FIG. 9B) resulted in rapid weight gain, resulting in up to 30% increase in body weight by 90 days (P<0.001), despite no change in food intake (FIGS. 14A and 14B). This was associated with significantly raised glycated hemoglobin levels (P<0.05) in both groups compared with control mice fed normal diet (FIG. 9D), as shown in Table 1, below.

TABLE 1
Effects of Daily (Pro³)GIP Administration on Glycated
Hemoglobin in Normal (TO) Mice Fed High Fat, High
Carbohydrate, Cafeteria and Normal Diets

Glycated Hemoglobin (%)

| Diet | Control | (Pro³)GIP |
|---|---|---|
| Normal | 3.8 ± 0.14 | 3.9 ± 0.14 |
| High Fat | 5.1 ± 0.48^{Δ} | 4.0 ± 0.21* |
| High Carbohydrate | 4.1 ± 0.22 | 4.3 ± 0.12 |
| Cafeteria | 5.2 ± 0.28^{Δ} | 4.0 ± 0.32* |

Glycated hemoglobin was measured 90 days after treatment with saline or (Pro³)GIP (25 nmol/kg bw/day) in groups of normal mice given access ad libitum to high fat diet, high carbohydrate diet, cafeteria diet and normal rodent diet. Values are means ± SEM for 8-10 mice.
*P < 0.05 compared with respective control on same diet.
^{Δ}P < 0.05 control group on normal diet.

Administration of (Pro³)GIP (▪) countered body weight gain in these mice and prevented elevation of glycated hemoglobin above control values (Δ). Mice fed high carbohydrate diet (FIG. 9C) neither displayed increased body weight gain or disturbances in blood glucose control. Administration of (Pro³)GIP did not affect the parameters measured in mice receiving high carbohydrate or normal diets.

### Example 8

### Additional Glu³-Substituted Peptide Analogues—Experimental Methods

Additional Glu³-substituted GIP analogues were synthesized, and their biological effects studied in this example, through Example 13.

Synthesis, Purification and Characterization of position 3 Substituted GIP Analogues. Native GIP and GIP analogues were sequentially synthesized on an Applied Biosystems automated peptide synthesizer (Model 432 A). Peptides were purified by reversed-phase HPLC on a Waters Millenium 2010 chromatography system (Software version 2.1.5) and subsequently characterized using electrospray ionization mass spectrometry (ESI-MS).

Animals. Obese diabetic (ob/ob) mice derived from the colony maintained at Aston University, UK (Bailey C. J. et al., 1982, Int. J. Obes. 6:11-21) were used at 14-18 weeks of age. Animals were age-matched, divided into groups and housed individually in an air-conditioned room at 22±2° C. with a 12 hour light: 12 hour dark cycle (08:00-20:00 hr). Drinking water and a standard rodent maintenance diet (Trouw Nutrition, Cheshire, UK) were freely available. All animal experiments were carried out in accordance with the UK Animals (Scientific Procedures) Act 1986.

Tissue Culture. Chinese Hamster Lung (CHL) fibroblast cells stably transfected with the human GIP-R were cultured in DMEM tissue culture medium containing 10%. (v/v) fetal bovine serum, 1% (v/v) antibiotics (100 U/ml penicillin, 0.1 mg/ml streptomycin) (all from Gibco, Paisley, Strathclyde, Scotland). BRIN-BD11 cells were cultured in RPMI-1640 tissue culture medium containing 10% (v/v) fetal bovine serum, 1% (v/v) antibiotics (100 U/ml penicillin, 0.1 mg/ml streptomycin) and 11.1 mm glucose. The origin and secretory characteristics of these cells have been described in detail previously (McClenaghan, N. H. et al., 1996, Diabetes 45:1132-1140). The cells were maintained in sterile tissue culture flasks (Corning Glass Works, Sunderland, UK) at 37° C. in an atmosphere of 5% CO₂ and 95% air using a LEEC incubator (Laboratory Technical Engineering, Nottingham, UK).

Biochemical analysis. Plasma glucose was assayed by an automated glucose oxidase procedure (Stevens 1973) using a Beckman Glucose Analyzer II (Beckman Instruments, Galway, Ireland). Plasma insulin was assayed by radioimmunoassay.

Statistics. Data are expressed as means±S.E. and the values compared using the Student's unpaired t-test. Where appropriate, data were compared using repeated measures ANOVA or one-way ANOVA, followed by the Student-Newman-Keuls post-hoc test. Groups of data were considered to be significantly different if p<0.05. Integrated glucose and insulin responses were calculated by the trapezoidal method, using the algorithm included in the software package Prism (version 3.02; GraphPad, San Diego, Calif.), with basal levels as base line.

### Example 9

### Degradation of GIP and Glu³-Substituted GIP Analogs by DPP-IV

This study looked at the percentage of the intact peptide analogue remaining after incubation with DPP IV.

Methods. GIP and related peptides (15 μg) were incubated (n=3) at 37° C. with DPP-IV (5 mU) for 0, 2, 4 and 8 hours in 50 mm triethanolamine-HCl (500 μl), pH 7.8, (final peptide concentration 2 μm). Enzymatic reactions were terminated by the addition of 10 μl of 10% (v/v) TFA/H₂O and stored at −20° C. prior to HPLC analysis as described previously (Gault, V. A. et al. 2002, Biochem. J. 367:913-920). The absorbance was monitored at 206 nm on a Spectra System UV2000 detector (Thermoquest Limited, Manchester, UK). The results are shown in Table 2, below.

TABLE 2
Percentage intact peptide remaining and estimated half-life
of GIP and related analogs after incubation with DPP-IV.

| | Intact Peptide Remaining (%) | | | | Estimated |
|---|---|---|---|---|---|
| Peptide | 0 hrs. | 2 hrs. | 4 hrs. | 8 hrs. | Half-Life |
| GIP | 100 | 20.9 ± 1.6 | 4.9 ± 1.2 | 0 | 1.3 |
| (Ala³)GIP | 100 | 5.9 ± 1.7* | 2.7 ± 0.1 | 0 | 1.1 |
| (Lys³)GIP | 100 | 41.4 ± 0.3** | 14.9 ± 1.1* | 0 | 1.7 |
| (Phe³)GIP | 100 | 15.0 ± 5.2 | 2.5 ± 0.1 | 0 | 1.2 |
| (Trp³)GIP | 100 | 22.5 ± 1.7 | 4.7 ± 2.4 | 0 | 1.3 |
| (Tyr³)GIP | 100 | 34.1 ± 4.1 | 6.3 ± 3.1 | 0 | 1.5 |
| (Hyp³)GIP | 100 | 100*** | 100*** | 100*** | >8 |
| (Pro³)GIP | 100 | 100*** | 100*** | 100*** | >8 |
| (Hyp³)LysPAL¹⁶GIP | 100 | 100*** | 100*** | 100*** | >8 |
| (Pro³)LysPAL¹⁶GIP | 100 | 100*** | 100*** | 100*** | >8 |

Data represent the percentage of intact peptide remaining (following HPLC separation) relative to the major degradation fragment GIP(3-42) after incubation with DPP-IV. The reactions were performed in triplicate and the results expressed as means ± S.E.
*p < 0.05,
**p < 0.01,
***p < 0.001 compared to native GIP.

As shown in Table 2, native GIP was rapidly hydrolyzed by DPP-IV with complete degradation by 8 hours. (Ala³)GIP was least resistant to DPP-IV, with almost complete degradation occurring after just 2 hours. (Phe³)GIP, (Trp³)GIP and (Tyr³)GIP displayed similar degradation profiles to the native peptide. (Lys³)GIP was significantly more resistant to DPP-IV. (Hyp³)GIP, (Pro³)GIP and their LysPAL¹⁶ counterparts were completely resistant to DPPIV degradation.

### Example 10

### Cyclic AMP Stimulation by Glu³-Substituted Peptide Analogues

This example studied the cAMP production in GIP-R transfected CHL cells. The results are shown in Table 3, below.

Methods. Receptor activation by GIP and GIP analogs in CHL cells transfected with the human GIP-R was according to published methodologies (Gault, V. A. et al. 2002, Biochem. J. 367:913-920). Briefly, GIP-R transfected CHL cells seeded into 24-well plates (Nunc, Roskilde, Denmark) at a density of 3.0×10⁵ cells per well were loaded with tritiated adenine (2 μCi; TRK311; Amersham, Buckinghamshire, UK) and allowed to grow for 18 hours at 37° C. The culture medium was removed and cells subsequently washed twice with 2 ml ice-cold HBS buffer (130 mm NaCl, 20 mm HEPES, 0.9 mm NaHPO₄, 0.8 mm MgSO₄, 5.4 mm KCl, 1.8 mm CaCl₂, 5.6 mm glucose and 25 μm phenol red) (pH 7.4). The cells were then exposed for 20 min at 37° C. to forskolin (10 μm; Sigma, Poole, Dorset, UK) or GIP/GIP analogs (10⁻¹³ to 10⁻⁷ m) in the presence or absence of native GIP (10⁻⁷ m) in HBS buffer containing 1 mm IBMX (Sigma, Poole, Dorset, UK). The medium was subsequently removed and the cells lysed with 11 ml of lysing solution (5% TCA, 3% SDS, 92% H₂O, also containing 0.1 mm unlabelled cAMP and 0.1 mm unlabelled ATP) (Sigma, Poole, Dorset, UK). The plates were then left on a shaker at room temperature for 30 minutes and tritiated cAMP formation determined by column chromatography using Dowex and alumina ion exchange columns (BioRad Life Science Research, Alpha Analytical, Larne, UK) as previously described (Gault, V. A. et al 2002, Biochem. J. 367:913-920).

Cyclic AMP stimulation of GIP analogs in receptor-transfected CHL cells is shown in Table 3, below.

TABLE 3
Effects of GIP analogs on cyclic AMP production and insulin secretion in vitro.

| | Cyclic AMP | | Insulin Secretion | |
|---|---|---|---|---|
| | | cAMP response | | Insulin response |
| | | in presence of | Maximal insulin | in presence of |
| | | 10⁻⁷ M GIP (% | response (% | 10⁻⁷ M GIP (% |
| Peptide | EC₅₀ (nm) | GIP max) | GIP max) | GIP max) |
| GIP | 0.5 | 100 | 100 | 100 |
| (Ala³)GIP | 2.3 | 88 ± 3 | 111 ± 6** | 115 ± 3 |
| (Lys³)GIP | 2.6 | 63.5 ± 4* | 78 ± 3* | 78 ± 4* |
| (Phe³)GIP | 1.0 | 62 ± 3* | 75 ± 3** | 73 ± 3* |
| (Trp³)GIP | 3.0 | 78 ± 2 | 73 ± 2** | 100 ± 5 |
| (Tyr³)GIP | 1.5 | 76 ± 4 | 75 ± 4* | 93 ± 2 |
| (Hyp³)GIP | 1340*** | 56 ± 4** | 56 ± 7** | 71 ± 7* |
| (Pro³)GIP | 207*** | 66 ± 7** | 53 ± 5*** | 64 ± 2** |
| (Hyp³)LysPAL¹⁶GIP | 138*** | 48 ± 4*** | 64 ± 5** | 62 ± 3** |
| (Pro³)LysPAL¹⁶GIP | 870*** | 63 ± 5** | 48 ± 4*** | 61 ± 3** |

Cyclic AMP production and insulin releasing activity were measured in GIP-R transfected CHL cells and glucose-responsive BRIN-BD11 cells, respectively. Data represent mean ± S.E. (n ≧ 3) and
*p < 0.05,
**p < 0.01,
***p < 0.001 compared to native GIP.

Native GIP dose-dependently (10⁻¹³ to 10⁻⁷ m) stimulated cyclic AMP production with an EC₅₀ value of 0.5 nm. In contrast, all of the Glu³-substituted analogs tested displayed weaker cyclic AMP activation responses with increased EC₅₀ values (1.0 to 294.5 mM; p<0.001). When incubated in the presence of a stimulatory concentration of GIP (10⁻⁷ m), (Ala³)GIP, (Trp³)GIP and (Tyr³)GIP did not significantly inhibit cAMP production. However, (Lys³)GIP, (Phe³)GIP and (Pro³)GIP significantly inhibited (p<0.05 to p<0.01) GIP-stimulated cAMP production (Table 3). (Pro³)GIPLysPAL¹⁶, (Hyp³)GIP and (Hyp³)GIP LysPAL¹⁶ similarly inhibited GIP-stimulated cAMP production (Table 3).

### Example 11

### Cellular Insulin Secretion of Glu³-Substituted Peptide Analogues

This example studied the insulin releasing activity in glucose-responsive BRIN-BD11 cells. The results are shown in Table 3, above.

Methods. Insulin release from BRIN-BD11 cells was determined by use of cell monolayers as described previously (Gault, V. A. et al. 2002, Biochem. J. 367:913-920). In brief, BRIN-BD11 cells were seeded into 24-well plates (Nunc, Roskilde, Denmark) at a density of 1.5×10⁵ cells per well and allowed to attach overnight in RPMI-1640 culture medium at 37° C. Acute tests for insulin secretion were preceded by 40 minutes pre-incubation at 37° C. in 1.0 ml Krebs Ringer Bicarbonate Buffer (KRBB, 115 mm NaCl, 4.7 mm KCl, 1.28 mm CaCl₂, 1.2 mM MgSO₄, 1.2 mm KH₂PO₄, 25 mM HEPES and 10 mm NaHCO₃; pH 7.4 with NaOH) supplemented with 0.1% (w/v) BSA and 1.1 mM glucose. Test incubations were performed (n=8) in the presence of 5.6 mm glucose over a range of concentrations (10⁻¹³ to 10⁻⁷ m) of GIP/GIP analogs in the presence or absence of native GIP (10⁻⁷ m). After 20 minutes incubation, the buffer was removed and used for measurement of insulin by radioimmunoassay (Flatt, P. R. and Bailey, C. J., 1981, Diabetologia 5:573-577).

The effects of GIP and GIP analogs on insulin secretion from clonal pancreatic BRIN-BD11 cells are summarized in Table 3, above. GIP dose-dependently (10⁻¹⁰ to 10⁷ m) stimulated insulin secretion (1.2- to 1.6-fold; p<0.05 to p<0.01) compared with control incubations (5.6 mm glucose alone). Of the analogs tested only (Ala³)GIP, at 10⁻⁷ m, elicited a significantly enhanced (1.7-fold; p<0.001) insulin response compared with control. When incubated in the presence of stimulatory GIP (10⁻⁷ m), (Ala³)GIP, (Trp³)GIP and (Tyr³)GIP did not significantly affect GIP-induced insulin secretion (Table 3). (Lys³)GIP, (Phe³)GIP, (Hyp³)GIP and (Pro³)GIP significantly inhibited p<0.05 to p<0.01) GIP-stimulated insulin secretion, illustrating their action as GIP antagonists. LysPAL¹⁶ derivatives of the two latter analogues similarly antagonized GIP-stimulated insulin secretion (Fable 3).

### Example 12

### Antihyperglycaemic and Insulin Releasing Activities of Glu³-Substituted Peptide Analogues

This example examined the effects of the peptide analogues on antihyperglycaemic action and insulin release with administered with glucose to ob/ob mice.

Methods. Plasma glucose and insulin responses were evaluated using 14-18 week old obese diabetic ob/ob mice (Bailey C. J. et al., 1982, Int. J. Obes. 6:11-21) following ip injection of native GIP or GIP analogs (25 nmol/kg body weight) immediately following the combined injection of GIP (25 nmoles/kg bw) together with glucose (18 mmol/kg bw). All test solutions were administered in a final volume of 5 ml/kg body weight. Blood samples were collected from the cut tip of the tail vein of conscious mice into chilled fluoride/heparin coated glucose microcentrifuge tubes (Sarstedt, Numbrecht, Germany) immediately prior to injection and at 15, 30 and 60 minutes post injection. Blood samples were immediately centrifuged using a Beckmnan microcentrifuge (Beckman Instruments, High Wycombe, Buckinghamshire, UK) for 30 seconds at 13,000×g. Plasma glucose was assayed using a Beckman Glucose Analyzer n (Beckman Instruments, High Wycombe, Buckinghamshire, UK) (Stevens, J. F., 1971, Clin. Chim. Acta 32:199-201) and plasma insulin was determined by RIA (Flatt, P. R. and Bailey, C. J., 1981, Diabetologia 5:573-577). All animal studies were carried out in accordance with the UK Animals (Scientific Procedures) Act 1986.

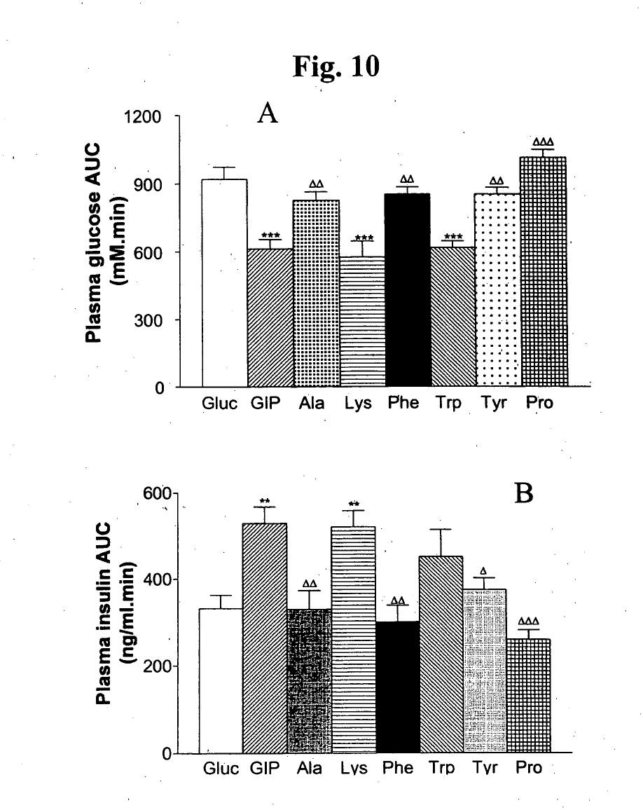

The results are shown in FIGS. 10A and 10B, which are a pair of bar graphs showing plasma glucose AUC (FIG. 10A) and plasma insulin AUC (FIG. 10B). Plasma glucose and insulin AUC values for 0-60 minutes post-injection are shown. Data are expressed as mean±S.E. for 8 mice. *p<0.05, **p<0.01, ***p<0.001 compared with glucose alone. ^{Δ}p<0.05, ^{ΔΔ}p<0.01, ^{ΔΔΔ}p<0.001 compared with native GIP.

Compared with i.p glucose alone (18 mmol/kg bw), administration of GIP (50 mmoles/kg bw) decreased the glycaemic excursion and enhanced insulin response. When administered together with native GIP, (Lys³)GIP and (Trp³)GIP did not significantly alter the relative plasma glucose and insulin profiles compared to native GIP. However, (Ala³)GIP, (Phe³)GIP, (Tyr³)GIP and (Pro³)GIP significantly inhibited the action of GIP and blood glucose levels were significantly elevated and insulin values decreased compared with GIP alone. These four analogues therefore represent effective antagonists of GIP.

### Example 13

### Longer-Term In Vivo Studies of the Glu³-Substituted Peptide Analogues

This example studied the longer-term effects of administration of (Pro³)GIP, (Hyp³)GIP, (Pro³)GIPLysPAL¹⁶ or (Hyp³)GIPLysPAL¹⁶ in ob/ob mice.

Methods. Ob/ob mice received, over an 14-day period, once daily i.p. injections (17:00 h) of either saline vehicle (0.9% (w/v), NaCl), (Pro³)GIP, (Hyp³)GIP, (Pro³)GIPLysPAL¹⁶ or (Hyp³)GIPLysPAL¹⁶ (25 mmol/kg body wt). Intraperitoneal glucose tolerance (18 mmol/kg body wt) and insulin sensitivity (50 U/kg body wt) tests were performed at the end of the study period. Blood samples taken from the cut tip of the tail vein of conscious mice at the times indicated in the Figures were immediately centrifuged using a Beckman microcentrifuge (Beckman Instruments, UK) for 30 seconds at 13,000×g. The resulting plasma was then aliquoted into fresh Eppendorf tubes and stored at −20° C. prior to glucose and insulin determinations.

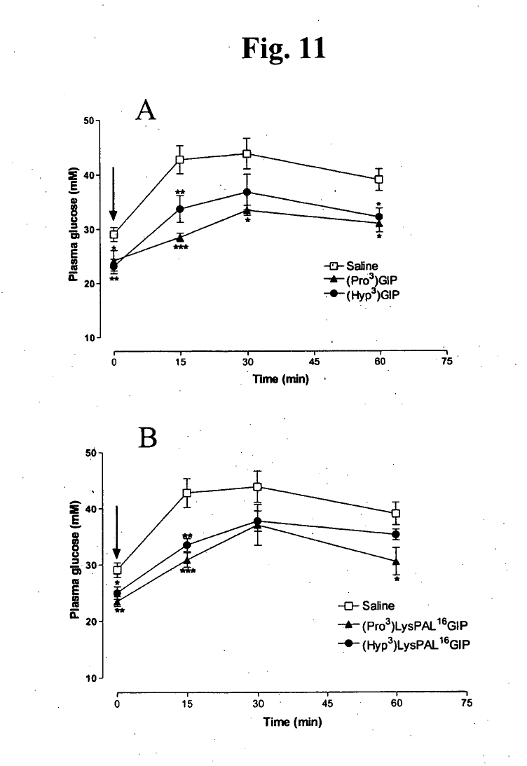

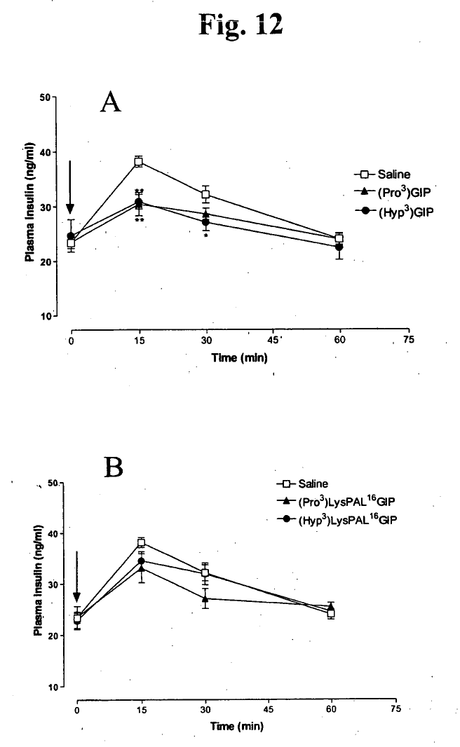

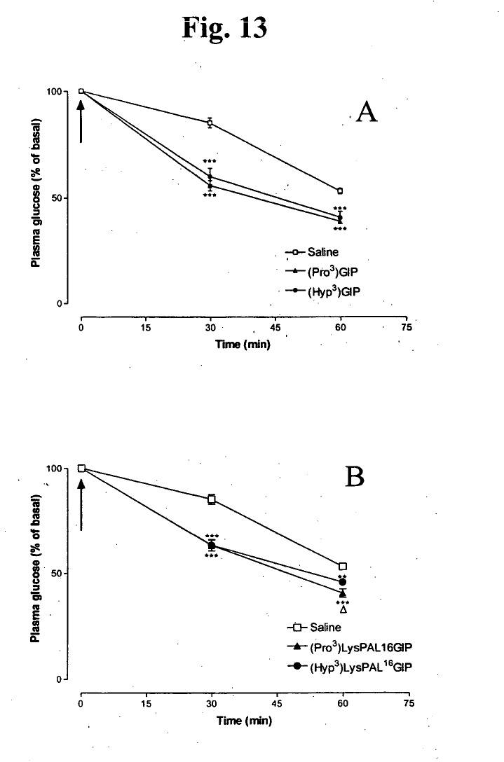

The results are shown in FIGS. 11-13, are pairs of line graphs showing glucose tolerance (FIGS. 11A, 11B), insulin response (FIGS. 12A, 12B) and insulin sensitivity (FIGS. 13A, 13B) in ob/ob mice following 14 once-daily injections of saline, (Pro³)GIP or (Hyp³)GIP (FIGS. 11A, 12A, 13B) or saline, (Pro³)LysPAL¹⁶GIP and (Hyp³)LysPAL¹⁶GIP (FIGS. 11B, 12B, 13B).

As shown in FIG. 11, daily administration of GIP analogues for 14 days resulted in significantly reduced (P<0.001) plasma glucose concentrations at 0, 15, 30 and 60 minutes following intraperitoneal glucose. Plasma insulin concentrations were generally reduced throughout the test (FIG. 12), indicative of induction of enhanced insulin sensitivity by administration of GIP peptides. Consistent with this view, the hypoglycemic action of insulin was significantly augmented in the various groups of mice treated daily with (Pro³)GIP, (Hyp³)GIP, (Pro³)GIPLysPAL¹⁶ or (Hyp³)GIPLysPAL¹⁶ (FIG. 13).

### Example 14

### Administration of (Pro³)GIP to Normal Mice Previously Fed High Fat Diet for 160 Days

This example shows the effects of the effects of daily (Pro³)GIP administration on body weight (A) and food intake (B) of Swiss TO mice fed a high fat diet for 160 days prior to commencement of treatment. The results are shown in FIGS. 15A-B, which are a set of line graphs. Normal mice were fed the high fat diet indicated above ad libitum from 6-8 weeks of age. Treated groups of mice received intraperitoneal injection of ProGIP (25 nmoles/kg body weight) each day for a further 60 days while receiving the same high fat diet. Mice administered (Pro³)GIP clearly promotes body weight loss (P<0.001) which is sustained and not associated with significant decrease in energy intake.

### Example 15

### Administration of (Pro³)GIP to Young Ob/Ob Mice Receiving Normal Diet

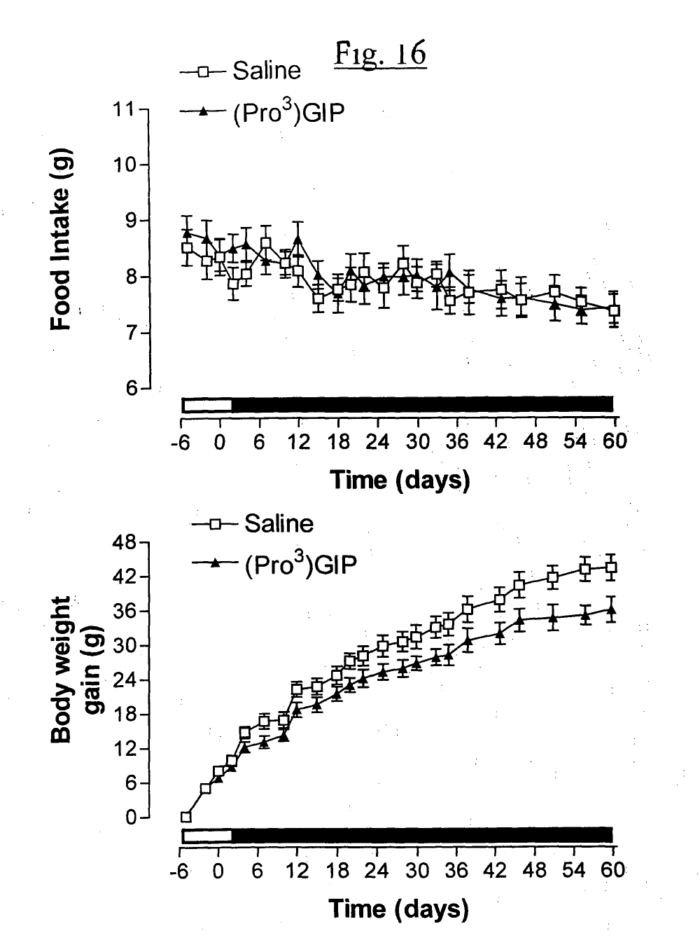

This example shows the effects of the effects of daily (Pro³)GIP administration on (A) food intake and body weight (B) of 5-7 weeks old ob/ob mice. The results are shown in FIGS. 16A-B, which are a set of line graphs. Ob/ob mice were fed a standard maintenance diet. Treated groups of mice received intraperitoneal injection of Pro³GIP (25 nmoles/kg body weight) each day for the 60 day duration of the experiment. (Pro³)GIP administration resulted in progressive decrease in body weight gain without changing food intake.

While this invention has been particularly shown and described with references to preferred embodiments thereof, it will be understood by those skilled in the art that various changes in form and details may be made therein without departing from the scope of the invention encompassed by the appended claims.

1. A method of:
(a) decreasing or preventing obesity; or
(b) preventing weight gain and promoting weight loss, the method comprising: administering a peptide analogue of GIP wherein the peptide analogue is at least 12 amino acid residues from the N-terminal end of GIP(1-42) and wherein the peptide analogue comprises an amino acid substitution or modification at position 3.

2. The method as claimed in claim 1, the peptide analogue is at least 12 amino acid residues from the N-terminal end of human GIP(1-42).

3. The method as claimed in claim 1, wherein the peptide analogue further comprises an amino acid substitution and/or amino acid modification, wherein, optionally, the peptide analogue further comprises an amino acid substitution and/or amino acid modification at one or both of positions 1 and 2.

4. The method as claimed in claim 3, wherein the, or each, amino acid substitution is selected from a D-amino acid substitution at position 1, a D-amino acid substitution at position 2, and a D-amino acid substitution at position 3.

5. The method as claimed in claim 4, wherein the amino acid at the 3 position is substituted by lysine, serine, proline, hydroxyproline, alanine, phenylalanine, tryptophan, tyrosine, 4-amino butyric acid (Abu), amino isobutyric acid (Aib), or sarcosine and wherein, optionally, the amino acid at position 2 is substituted by lysine, serine, proline, hydroxyproline, alanine, phenylalanine, tryptophan, tyrosine, 4-amino butyric acid (Abu), amino isobutyric acid (Aib), or sarcosine.

6. The method as claimed in claim 5, wherein the peptide analogue is selected from (Lys³)GIP, (Ser³)GIP, (Pro³)GIP, (Hyp³)GIP, (Ala³)GIP, (Phe³)GIP, (Trp³)GIP, (Tyr³)GIP, (Abu³)GIP, (Aib³)GIP, (Aib)³GIP and (Sar³)GIP.

7. The method as claimed in claim 1, wherein the peptide analogue is further modified at position 1, the modification being selected from N-terminal alkylation, N-terminal acetylation, N-terminal acylation, the addition of an N-terminal isopropyl group, the addition of an N-terminal pyroglutamic acid, and the addition of an N-terminal polyethylene glycol (PEG) molecule.

8. The method as claimed in claim 1, wherein the peptide analogue is selected from N-Ac(Lys³)GIP, N-Ac(Ser³)GIP, N-Ac(Pro³)GIP, N-Ac(Hyp³)GIP, N-Ac(Ala³)GIP, N-Ac(Phe³)GIP, N-Ac(Trp³)GIP, N-Ac(Tyr³)GIP, N-Ac(Abu³)GIP, N-Ac(Aib³)GIP and N-Ac(Sar³)GIP.

9. The method as claimed in claim 1, wherein the peptide analogue comprises a modification by an acyl radical addition, optionally a fatty acid addition, at an epsilon amino group of at least one lysine residue.

10. The method as claimed in claim 9, wherein the modification is the linking of a C-8 octanoyl group, a C-10 decanoyl group, a C-12 lauroyl group, a C-14 myristoyl group, a C-16 palmitoyl group, a C-18 stearoyl group, or a C-20 acyl group to the epsilon amino group of a lysine residue.

11. The method as claimed in claim 10, wherein the modification is the linking of a C-16 palmitoyl group to a lysine residue selected from the group consisting of Lys¹⁶, Lys³⁰, Lys³², Lys³³ and Lys³⁷.

12. The method as claimed in claim 11, wherein the peptide analogue is selected from (Lys³)GIP(LysPAL¹⁶), (Lys³)GIP(LysPAL³⁷), N-Ac(Lys³)GIP(LysPAL¹⁶), N-Ac(Lys³)GIP(LysPAL³⁷), (Ser³)GIP(LysPAL¹⁶), (Ser³)GIP(LysPAL³⁷), N-Ac(Ser³)GIP(LysPAL¹⁶), N-Ac(Ser³)GIP(LysPAL³⁷), (Pro³)GIP(LysPAL¹⁶), (Pro³)GIP(LysPAL³⁷), N-Ac(Pro³)GIP(LysPAL¹⁶), N-Ac(Pro³)GIP(LysPAL³⁷), (Hyp³)GIP(LysPAL¹⁶), (Hyp³)GIP(LysPAL³⁷), N-Ac(Hyp³)GIP(LysPAL¹⁶), N-Ac(Hyp³)GIP(LysPAL³⁷), (Ala³)GIP, (Ala³)GIP(LysPAL¹⁶), (Ala³)GIP(LysPAL³⁷), N-Ac(Ala³)GIP(LysPAL¹⁶), N-Ac(Ala³)GIP(LysPAL³⁷), (Phe³)GIP(LysPAL¹⁶), (Phe³)GIP(LysPAL³⁷), N-Ac(Phe³)GIP(LysPAL¹⁶), N-Ac(Phe³)GIP(LysPAL³⁷), (Trp³)GIP(LysPAL¹⁶), (Trp³)GIP(LysPAL³⁷), N-Ac(Trp³)GIP(LysPAL¹⁶), N-Ac(Trp³)GIP(LysPAL³⁷), (Tyr³)GIP(LysPAL¹⁶), (Tyr³)GIP(LysPAL³⁷), N-Ac(Tyr³)GIP(LysPAL¹⁶), N-Ac(Tyr³)GIP(LysPAL³⁷), (Abu³)GIP(LysPAL¹⁶), (Abu³)GIP(LysPAL³⁷), N-Ac(Abu 3)GIP(LysPAL¹⁶), N-Ac(Abu³)GIP(LysPAL³⁷), (Aib³)GIP(LysPAL¹⁶), (Aib³)GIP(LysPAL³⁷), N-Ac(Aib³)GIP(LysPAL¹⁶), N-Ac(Aib³)GIP(LysPAL³⁷), (Sar³)GIP(LysPAL¹⁶), (Sar³)GIP(LysPAL³⁷), N-Ac(Sar³)GIP(LysPAL¹⁶), and N-Ac(Sar³)GIP(LysPAL³⁷).

13. The method as claimed in claim 11, wherein the method is for decreasing or preventing obesity.

14. The method as claimed in claim 11, wherein the method is for preventing weight gain and promoting weight loss.

15. The method as claimed in claim 1, wherein the peptide analogue is covalently attached to a polyethylene glycol (PEG) molecule.

16. The method as claimed in claim 1, wherein the medicament further comprises a pharmaceutically acceptable carrier.

17. The method as claimed in claim 1, wherein the peptide analogue is in the form of a pharmaceutically acceptable salt.

18. The method as claimed in claim 1, wherein the peptide analogue is in the form of a pharmaceutically acceptable acid addition salt.

19. The method as claimed in claim 1, wherein the medicament further comprises an agent having an antidiabetic and/or antiobesity effect.

20. A peptide analogue of GIP(1-42), wherein the peptide analogue is at least 12 amino acid residues from the N-terminal end of GIP(1-42) and wherein the peptide analogue comprises at least one amino acid substitution or modification at position 3 and wherein the amino acid at the 3 position can be substituted by any L-amino acid selected from L-alanine, L-arginine, L-asparagine, L-aspartic acid, L-cysteine, L-glutamine, L-glycine, L-histidine, L-isoleucine, L-leucine, L-lysine, L-methionine, L-phenylalanine, L-proline, L-serine, L-threonine, L-tryptophan, L-tyrosine and L-valine or by any D-amino acid selected from by D-arginine, D-asparagine, D-aspartic acid, D-cysteine, D-glutamine, D-glycine, D-histidine, D-isoleucine, D-leucine, D-lysine, D-methionine, D-phenylalanine, D-proline, D-serine, D-threonine, D-tryptophan, D-tyrosine and D-valine or by any other L- or D-amino acid other than those commonly encountered in the genetic code, including beta amino acids such as beta-alanine and omega amino acids such as 3-amino propionic, 4-amino butyric, etc, ornithine, citrulline, homoarginine, t-butyl alanine, t-butyl glycine, N-methyl isoleucine, phenylglycine, cyclohexylalanine, norleucine, cysteic acid, and methionine sulfoxide.

21. The peptide analogue as claimed in claim 20, wherein the peptide analogue comprises an amino acid modification by fatty acid addition at an epsilon amino group of said at least one lysine residue.

22. The peptide analogue as claimed in claim 20, wherein the peptide analogue is modified at position 1 by N-terminal alkylation, N-terminal acetylation, N-terminal C₆₋₂₀ acylation, the addition of an N-terminal isopropyl group, the addition of an N-terminal pyroglutamic acid, or the addition of an N-terminal polyethylene glycol (PEG) molecule.

23. The peptide analogue as claimed in claim 20 and selected from the group comprising (Lys³)GIP(LysPAL¹⁶), (Lys³)GIP(LysPAL³⁷), N-Ac(Lys³)GIP(LysPAL¹⁶), N-Ac(Lys³)GIP(LysPAL³⁷), (Ser³)GIP(LysPAL¹⁶), (Ser³)GIP(LysPAL³⁷), N-Ac(Ser³)GIP(LysPAL¹⁶), N-Ac(Ser³)GIP(LysPAL³⁷), (Pro³)GIP(LysPAL¹⁶), (Pro³)GIP(LysPAL³⁷), N-Ac(Pro³)GIP(LysPAL¹⁶), N-Ac(Pro³)GIP(LysPAL³⁷), (Hyp³)GIP(LysPAL¹⁶), (Hyp³)GIP(LysPAL³⁷), N-Ac(Hyp³)GIP(LysPAL¹⁶), N-Ac(Hyp³)GIP(LysPAL³⁷), (Ala³)GIP, (Ala³)GIP(LysPAL¹⁶), (Ala³)GIP(LysPAL³⁷), N-Ac(Ala³)GIP(LysPAL¹⁶), N-Ac(Ala³)GIP(LysPAL³⁷), (Phe³)GIP(LysPAL¹⁶), (Phe³)GIP(LysPAL³⁷), N-Ac(Phe³)GIP(LysPAL⁶), N-Ac(Phe³)GIP(LysPAL³⁷), (Trp³)GIP(LysPAL¹⁶), (Trp³)GIP(LysPAL³⁷), N-Ac(Trp³)GIP(LysPAL¹⁶), N-Ac(Trp³)GIP(LysPAL³⁷), (Tyr³)GIP(LysPAL¹⁶), (Tyr³)GIP(LysPAL³⁷), N-Ac(Tyr³)GIP(LysPAL¹⁶), N-Ac(Tyr³)GIP(LysPAL³⁷), (Abu³)GIP(LysPAL¹⁶), (Abu³)GIP(LysPAL³⁷), N-Ac(Abu³)GIP(LysPAL³⁷), N-Ac(Aib³)GIP(LysPAL³⁷), (Aib³)GIP(LysPAL³⁷), (Aib³)GIP(LysPAL³⁷), N-Ac(Aib³)GIP(LysPAL¹⁶), N-Ac(Aib³)GIP(LysPAL³⁷), (Sar³)GIP(LysPAL¹⁶), (Sar³)GIP(LysPAL³⁷), N-Ac(Sar³)GIP(LysPAL¹⁶) and N-Ac(Sar³)GIP(LysPAL³⁷).

24. The peptide analogue as claimed in claim 23, wherein the peptide analogue is selected from the group comprising (Ala³)GIP, (Ala³)GIP(LysPAL¹⁶), (Ala³)GIP(LysPAL³⁷), N-Ac(Ala³)GIP(LysPAL³⁷), N-Ac(Ala³)GIP(LysPAL³⁷), (Pro³)GIP(LysPAL¹⁶), (Pro³)GIP(LysPAL³⁷), N-Ac(Pro³)GIP(LysPAL¹⁶), N-Ac(Pro³)GIP(LysPAL³⁷), (Hyp³)GIP(LysPAL¹⁶), (Hyp³)GIP(LysPAL³⁷), N-Ac(Hyp³)GIP(LysPAL¹⁶) and N-Ac(Hyp³)GIP(LysPAL³⁷).

25. The peptide analogue as claimed in claim 23, wherein the peptide analogue is selected from the group comprising (Ala³)GIP, (Pro³)GIP(LysPAL¹⁶) and (Hyp³)GIP(LysPAL¹⁶).

26. The peptide analogue of GIP(1-42), wherein the peptide analogue is at least 12 amino acid residues from the N-terminal end of GIP(1-42) and wherein the peptide analogue comprises at least one amino acid substitution or modification at position 3 and wherein the peptide analogue further comprises a further amino acid substitution and/or a further amino acid modification, wherein said at least one modification or further modification is the addition of a polyethylene glycol (PEG) molecule.

27. The peptide analogue as claimed in claim 26, wherein said at least one modification is the addition of a polyethylene glycol (PEG) molecule at position 1.

28. The peptide analogue as claimed in claim 26, wherein said at least one modification is the addition of a polyethylene glycol (PEG) molecule at a position other than position 1.

29. The peptide analogue as claimed in claim 26, wherein the peptide analogue further comprises an amino acid substitution of cysteine for one or more of the residues and wherein the modification is the addition of a polyethylene glycol (PEG) molecule at said at least one substituted cysteine residue.

30. The peptide analogue as claimed in claim 26, wherein the peptide analogue further comprises an amino acid substitution of lysine for one or more of the residues and an amino acid modification by fatty acid addition at an epsilon amino group of said at least one substituted lysine residue.

31. A pharmaceutical composition comprising a peptide analogue as claimed in claim 20, in association with a pharmaceutically acceptable carrier.

32. A pharmaceutical composition comprising a peptide analogue as claimed in claim 26, in association with a pharmaceutically acceptable carrier.


SEQUENCE LISTING (6 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 42 aa, Sus scrofa
YAEGTFISDYSIAMDKIRQQDFVNWLLAQKGKKSDWKHNITQ

SEQ ID NO: 3 - protein, 42 aa, Bos taurus
YAEGTFISDYSIAMDKIRQQDFVNWLLAQKGKKSDWIHNITQ

SEQ ID NO: 4 - protein, 42 aa, Mesocricetus auratus
YAEGTFISDYSIAMDKIRQQDFVNWLLAQKGKKNDWKHNITQ

SEQ ID NO: 5 - protein, 42 aa, Rattus norvegicus
YAEGTFISDYSIAMDKIRQQDFVNWLLAQKGKKNDWKHNLTQ

SEQ ID NO: 6 - protein, 42 aa, Mus musculus
YAEGTFISDYSIAMDKIRQQDFVNWLLAQRGKKSDWKHNITQ